(19) United States
(12) Patent Application Publication
(10) Pub. No.: US 2025/0025440 A1
(43) Pub. Date: Jan. 23, 2025

(54) COMPOSITIONS AND METHODS FOR MANAGEMENT OF HUMAN PAPILLOMA VIRUS-ASSOCIATED CANCERS

(71) Applicant: LOMA LINDA UNIVERSITY, Loma Linda, CA (US)

(72) Inventors: Penelope J. DUERKSEN-HUGHES, Loma Linda, CA (US); Lennox CHITSIKE, Loma Linda, CA (US)

(73) Assignee: LOMA LINDA UNIVERSITY, Loma Linda, CA (US)

(21) Appl. No.: 18/546,642

(22) PCT Filed: Feb. 17, 2022
(86) PCT No.: PCT/US22/70714

§ 371 Date: Aug. 16, 2023

Related U.S. Application Data
(60) Provisional application No. 63/200,156, filed on Feb. 17, 2021.

(51) Int. Cl. A61K 31/352; A61K 33/243; A61K 39/395; A61N 5/10

(52) U.S. Cl. CPC A61K 31/352; A61K 33/243; A61K 39/3955; A61N 5/10; A61N 2005/1098

(57) ABSTRACT

Provided here are compositions and methods for management of human papillomavirus-associated cancers. One such method of treating a human papillomavirus-associated cancer in a subject includes administering to the subject a therapeutically effective amount of gambogic acid or 30-hydroxygambogic acid or a pharmaceutically acceptable derivative thereof. These methods can also include administering one or more of radiation or a chemotherapeutic agent.

### CROSS-REFERENCE TO RELATED APPLICATIONS

This application is a U.S. National Phase Under 35 U.S.C. § 371 of International Application No. PCT/US2022/070714, titled “COMPOSITIONS AND METHODS FOR MANAGEMENT OF HUMAN PAPILLOMA VIRUS-ASSOCIATED CANCERS,” filed Feb. 17, 2022, which claims the benefit of and priority to U.S. Provisional Application No. 63/200,156, filed on Feb. 17, 2021, the contents of all of which are incorporated by reference herein in their entirety.

### GOVERNMENT SUPPORT

This invention was made with Government support under Grant No. P20GM113117 awarded by the National Institute of General Medical Sciences of the National Institutes of Health and Grant No. R21NS73059 awarded by the National Institute of Neurological Disorders and Stroke of the National Institutes of Health. The Government has certain rights in the invention.

### TECHNICAL FIELD

This disclosure relates to compositions and methods for management of Human Papilloma Virus (HPV)-associated cancers.

### BACKGROUND

High-risk HPV causes several types of cancer, especially in the cervix, oropharynx, anus, penis, vagina, and vulva. HPV-related cancers include cervical cancer, oropharyngeal cancers, anal cancer, penile cancer, vaginal cancer, and vulvar cancer. HPV infects the squamous cells lining the inner surfaces of these organs, thus leading to squamous cell carcinoma.

Head and neck squamous cell carcinomas (HNSCC) are heterogeneous tumors that arise in the upper respiratory tract and are the sixth most common cancer worldwide by incidence. The two main subtypes of HNSCC—HPV⁻-HNSCC and HPV⁺-HNSCC—are distinct and diverging in their features. HPV⁻-HNSCC, historically caused by chemical carcinogens such as alcohol and tobacco, has been in decline for the past three decades. In parallel, HPV⁺-HNSCC, which is caused by HPV, has risen dramatically (over 225%) within the same period. Changes in sexual practices, particularly in Western countries, has increased the colonization of the oral mucosa by HPV, along with the associated malignancies. The advent of HPV vaccines has the potential to prevent this number from climbing upwards in the next decades. However, even with the availability of vaccines, the burden of HPV⁺-HNSCC remains a concern in the future due to limited uptake of the vaccines. The vaccine is ineffective in those already infected patients. For patients already presenting with HNSCC, current treatment guidelines recommend a combination approach involving surgery, radiation and chemotherapy, irrespective of the HPV status. This approach is not optimal, given the known distinct tumor biology and response to therapy. Compared to its HPV⁻ counterpart, HPV⁺-HNSCC carries a more favorable prognosis and is more prevalent in younger and otherwise healthier patients. Importantly, the use of such standard therapies is associated with debilitating life-long morbidities. Taken together, there has been a general consensus that the HPV⁺ subgroup may be over-treated and that selective therapies that spare patients from these long-term and deleterious side effects are needed.

Therapy of HNSCC patients is primarily based on cytotoxic treatments such as radiation and cisplatin and less on tumor biology-based agents as has been the trend for other cancers. However, radiotherapy is associated with severe long-term effects which are exacerbated by the toxicity of cisplatin. This problem stands out even more in HPV-associated HNSCC patients who generally respond better to therapy and have better prognosis. There is therefore a need for safer, less aggressive regimens or alternatives to cisplatin that maintain oncologic outcomes without compromising on the quality of life of patients. In addition, recent clinical trials have revealed that a significant number of HPV-associated patients have resistance (primary and/or acquired) to treatment with cetuximab, which may be the contributing factor to the observed inferiority of cetuximab to cisplatin in terms of efficacy. There is therefore also need for overcoming this cetuximab resistance.

### SUMMARY

Applicant has recognized an unmet and urgent need for management of HPV-related cancers. Disclosed herein are compounds and methods addressing the shortcomings of the art, and may provide any number of additional or alternative advantages, including compositions and methods for management of HPV-related cancers based on HPV status.

An embodiment of the method of treating a HPV-related cancer in a subject includes the step of administering to the subject a therapeutically effective amount of gambogic acid or 30-hydroxygambogic acid or a pharmaceutically acceptable derivative thereof. The subject can have cervical cancer, oropharyngeal cancer, anal cancer, penile cancer, vaginal cancer, or vulvar cancer. In an embodiment, the subject has head and neck squamous cell carcinoma. The method can further include administering to the subject a therapeutically effective amount of radiation prior to the administration of the therapeutically effective amount of gambogic acid or a pharmaceutically acceptable derivative thereof. The method can further include administering to the subject a therapeutically effective amount of radiation subsequent to the administration of the therapeutically effective amount of gambogic acid or a pharmaceutically acceptable derivative thereof. The method can further include administering to the subject a therapeutically effective amount of an apoptosis-activating chemotherapeutic agent. The method can further include administering to the subject a therapeutically effective amount of one or more of cisplatin, cetuximab, gefitinib, erlotinib, afatinib, dacomitinib, osimertinib, rociletinib, omultinib, and doxorubicin. In certain embodiments, the treatment regimen can also include radiation (either photon or proton).

An embodiment of the method of treating a head and neck squamous cell carcinoma in a subject includes the step of administering to the subject a therapeutically effective amount of gambogic acid or 30-hydroxygambogic acid or a pharmaceutically acceptable derivative thereof, in addition to a therapeutically effective amount of at least one chemotherapeutic agent for activating apoptosis. In certain embodiments, the treatment regimen can also include radiation (either photon or proton). An embodiment of the method of treating a head and neck squamous cell carcinoma in a subject includes the step of administering to the subject a therapeutically effective amount of gambogic acid or 30-hydroxygambogic acid or a pharmaceutically acceptable derivative thereof, in addition to a therapeutically effective amount of one or more of cisplatin, cetuximab, gefitinib, erlotinib, afatinib, dacomitinib, osimertinib, rociletinib, omultinib, and doxorubicin. In certain embodiments, the treatment regimen can also include radiation (either photon or proton).

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. To facilitate this description, like reference numerals designate like structural elements or procedures in a method. Embodiments are illustrated by way of example and not by way of limitation in the FIGS. of the accompanying drawings.

FIG. 1 is a diagrammatic representation of the high content screening strategy for certain embodiments. Screening funnel scheme shows the screening activity performed at each given step of hit compound triage and the respective decision criterion used for hit selection. Primary screening was followed by secondary screening and cheminformatics filtering using PAINS databases. Relationships between structure and activity were then analyzed before one candidate was chosen for cell studies.

FIGS. 2A-2C are graphical representations of three variables for assay optimization: the Z-factor, signal to background ratio (S/B), and the coefficient of variation (CV %). FIG. 2A is a graphical representation of Z factor. Scores were determined for each plate, and the median and average scores were found to both be >0.6, indicating suitability of the assay for high content screening. FIG. 2B is a graphical representation of the signal to background ratio for each plate. FIG. 2C is a scatter plot of the activity of the compounds (compounds above the solid green line were further analyzed). Hits were selected using a computed cut off value of % Inhibition against 3 standard deviations from the mean.

FIGS. 3A-3K are graphical representations of the selectivity profiles and indices of the selected 11 compounds. Binding activity graphs for the 11 compounds that passed both the 6xHis-GST and Caspase 8-Caspase 8 counter-screens are shown. The graphs show activity of each compound against Caspase 8-E6 and 6xHis-GST binding, and the calculated SI values from the activity against these two types of substrates (IC₅₀/IC₅₀). FIG. 3A is such graph for Compound #2, FIG. 3B is such graph for Compound #4, FIG. 3C is such graph for Compound #6, FIG. 3D is such graph for Compound #9, FIG. 3E is such graph for Compound #11, FIG. 3F is such graph for Compound #15, FIG. 3G is such graph for Compound #24, FIG. 3H is such graph for Compound #29, FIG. 3I is such graph for Compound #30, FIG. 3J is such graph for Compound #32, and FIG. 3K is such graph for Compound #38. FIG. 3L is a graphical representation of the interaction of these compounds with the dimerization of caspase 8.

FIGS. 4A-4B are graphical representations of the activity of gambogic acid as compared to myricetin in binding assays with caspase 8 and p53, respectively. FIG. 4A is a graphical representation of the head-to-head comparison of gambogic acid with myricetin, which has previously demonstrated activity against E6 binding to Caspase 8 and E6AP. FIG. 4B is a graphical representation of the head-to-head comparison of gambogic acid with myricetin, which has previously demonstrated activity against E6 binding to E6AP. The inhibitory activity of gambogic acid as measured by AlphaScreen™ is superior to that of myricetin.

FIG. 5 is an image showing the analogs of gambogic acid following structure-activity relationship analysis. Rings A, B, C, D make up the core scaffold of gambogic acid. Analog #1 is 30-hydroxygambogic acid (GA-OH), analog #2 is morellic acid, analog #3 is gambogenic acid, analog #4 is gambogic amide, analog #5 is neo-gambogic acid, analog #6 is gambogin, analog #7 is iso morellinol, and analog #8 is acetyl iso-gambogic acid.

FIGS. 6A-6B are graphical representations of the activity profiles of analogs of gambogic acid in vitro (AlphaScreen) and in vivo (HPV⁺ cell line assays). FIG. 6A is a graphical representation of the E6 hit analog activity in vitro using E6-Caspase 8 substrates. With the exception of #3, 5, and 6, most analogs show activity close to that of the parent compound. FIG. 6B is a graphical representation of the E6 hit analog activity in vivo (cells) context, #3, 5, and 6 also show low activity; #8 is a surprise addition to this group. #1 remains the most promising lead in terms of activity in the HPV⁺ cell line.

FIGS. 7A-7C are graphical representations of the sensitivity of HPV⁺ cell lines and HPV⁻ cell lines to GA-OH-mediated growth inhibition. FIG. 7A is a graphical representation of the cell growth inhibition of HNSCC HPV⁺ and HPV⁻ cell lines by GA-OH. FIG. 7B is a graphical representation of the cell growth inhibition of cervical cancer HPV⁺ and HPV⁻ cell lines by GA-OH. HPV⁺ cell lines display higher sensitivity than do HPV⁻ cell lines as shown by the leftwards shift of the HPV⁺ curves (SCC47, 90, 104, 152 in FIG. 7A and SiHa and CaSki in FIG. 7B. FIG. 7C is a graphical representation of the effects of GA-OH on the clonogenicity of the surviving fractions of HPV⁺ versus HPV⁻ cell lines. GA-OH displayed higher cytotoxicity to HPV⁺ cells. (+ and closed shapes represent HPV⁺ cell lines and − and open shapes the HPV⁻ cell lines.)

FIG. 8A is a photographic image of blots analyzing Caspase 8 expression and activation of downstream targets. SCC19, SCC90 and SCC104 were seeded and treated with vehicle, 0.5 μM and 1 μM Of GA-OH for 24 hours. Activation of p53 was tested by blotting for p53 expression and activation. Caspase 8 activation and apoptosis was tested by blotting for Caspase 8 expression and activation of downstream targets. FIG. 8B is a graphical representation of the Caspase 3/7 activity of gambogic acid in various HPV+ and HPV− cell lines. These cell lines were seeded in 96 well plates and treated with 0.75 μM GA-OH. Caspase 3/7 activity was measured after 24 hours. HPV⁺ cell lines show high levels of Caspase 3/7 activity.

FIGS. 9A-9E are graphical representations of the relative expression of cleaved PARP, p53, p21, cleaved caspase 3, and cleaved caspase 8, respectively in SCC19, SCC90 and SCC104 cell lines when treated with vehicle, or 0.5 μM, or 1 μM of GA-OH, each of the targets above using B-actin as a loading control is shown.

FIG. 10 is a graphical representation of the cell viability as measured by MTT ((3-[4,5-dimethylthiazol-2-yl]-2,5 diphenyl tetrazolium bromide), when three HPV⁺ cell lines (SCC090, SCC104, SiHa) and two HPV⁻ cell lines (SCC19, SCC84) were treated with gambogic acid.

FIGS. 11A-11D are graphical representations of the radio-response of HPV⁻ cell lines (SCC19 and SCC29) and HPV⁺ cell lines (SCC47 and SCC104) when subject to photon radiation followed by treatment with either the vehicle alone or cisplatin (CCDP), as measured by MTT assays. The dose enhancement ratio for 10% survival (DER₁₀) was calculated as follows: DER₁₀=(Dose correlated with 10% survival fraction in radiation-treated cells)/(Dose correlated with 10% survival fraction in radiation and inhibitor treated cells). DER10>1 indicates that that compound radiosensitizes cancer cells. FIGS. 11E-12H are graphical representations of the radio-response of HPV⁻ cell lines (SCC19 and SCC29) and HPV⁺ cell lines (SCC47 and SCC104) when subject to photon radiation followed by treatment with either the vehicle alone or cetuximab, as measured by MTT assays.

FIGS. 12A-12D are graphical representations of the radio-response of HPV⁻ cell lines ((SCC19 and SCC29) and HPV⁺ cell lines (SCC47 and SCC104) when subject to photon radiation followed by treatment with either the vehicle alone or GA-OH, as measured by MTT assays.

FIGS. 13A-13C are graphical representations of the radio-response of HPV⁺ cell line (SCC152) when subject to photon radiation followed by treatment with either the vehicle alone or cisplatin or cetuximab or GA-OH, as measured by MTT assays.

FIGS. 14A-14E are graphical representations of the extent of sensitization in HNSCC cell lines as measured by DER₁₀ when HPV⁻ cell lines (SCC19 and SCC29) and HPV⁺ cell lines (SCC47, SCC104, and SCC152) are subject to photon radiation followed by treatment with cisplatin or cetuximab or GA-OH.

FIGS. 15A-15B are graphical representations of the cell cycle analysis of SCC47 and SCC19 cell lines after irradiation and followed by treatment with control or with GA-OH, as measured by flow cytometry assays after 24 hours.

FIGS. 16A-16B are graphical representations of the fraction of G2-M arrested cells in SCC47 and SCC19 lines after irradiation and followed by treatment with control or with GA-OH, as measured by flow cytometry assays after 24 hours.

FIGS. 17A-17B are graphical representations of the kinetics of G2-M arrest when SCC47 and SCC19 are irradiated and followed by treatment with control or with GA-OH, as measured by flow cytometry assays after 24 hours.

FIGS. 18A-18B are graphical representations of the apoptotic analysis of SCC47 and SCC19 after irradiation with or without GA-OH after 24 hours, as measured by flow cytometry assays.

Numerous other aspects, features and benefits of the present disclosure may be made apparent from the following detailed description taken together with the formulae and tables.

### DETAILED DESCRIPTION

In the following description, numerous specific details are set forth in order to provide a thorough understanding of the various embodiments. In other instances, well-known processes and methods may not be described in particular detail in order not to unnecessarily obscure the embodiments described here. Additionally, illustrations of embodiments here may omit certain features or details in order to not obscure the embodiments described here.

In the following detailed description, reference is made to the accompanying formulae and tables that form a part of the specification. Other embodiments may be utilized, and logical changes may be made without departing from the scope of the disclosure. Therefore, the following detailed description is not to be taken in a limiting sense.

Disclosed herein are compositions and methods for management of a HPV-related cancer in a subject. An embodiment of the method of treating a HPV-related cancer includes the step of administering to the subject a therapeutically effective amount of gambogic acid or 30-hydroxygambogic acid or a pharmaceutically acceptable derivative thereof. The subject can have cervical cancer, oropharyngeal cancer, anal cancer, penile cancer, vaginal cancer, or vulvar cancer. The method can further include administering to the subject a therapeutically effective amount of radiation prior to the administration of the therapeutically effective amount of gambogic acid or a pharmaceutically acceptable derivative thereof. The method can further include administering to the subject a therapeutically effective amount of radiation subsequent to the administration of the therapeutically effective amount of gambogic acid or a pharmaceutically acceptable derivative thereof. The method can further include administering to the subject a therapeutically effective amount of an apoptosis-activating chemotherapeutic agent. Such a chemotherapeutic agent induces or initiates the apoptotic signaling pathway and leads to cell death or decreased proliferation. The method can further include administering to the subject a therapeutically effective amount of one or more of cisplatin, cetuximab, cisplatin, gefitinib, erlotinib, afatinib, dacomitinib, osimertinib, rociletinib, omultinib, and doxorubicin. In certain embodiments, the treatment regimen can also include radiation (either photon or proton).

Disclosed herein are compositions and methods for management of head and neck squamous cell carcinoma. An embodiment of the method of treating a head and neck squamous cell carcinoma in a subject includes the step of administering to the subject a therapeutically effective amount of gambogic acid or a pharmaceutically acceptable derivative thereof. The head and neck squamous cell carcinoma in this instance is positive for presence of human papillomavirus. An embodiment of the method of treating a head and neck squamous cell carcinoma in a subject includes the step of administering to the subject a therapeutically effective amount of 30-hydroxygambogic acid or a pharmaceutically acceptable derivative thereof. The head and neck squamous cell carcinoma in this instance is positive for presence of human papillomavirus. In certain embodiments, the treatment regimen can also include radiation (either photon or proton). An embodiment of the method of treating a head and neck squamous cell carcinoma in a subject includes the step of administering to the subject a therapeutically effective amount of 30-hydroxy gambogic acid or gambogic acid or a pharmaceutically acceptable derivative thereof, in addition to a therapeutically effective amount of at least one chemotherapeutic agent configured for activating apoptosis. In certain embodiments, the treatment regimen can also include radiation (either photon or proton).

An embodiment of the method of treating a head and neck squamous cell carcinoma in a subject includes the step of administering to the subject a therapeutically effective amount of 30-hydroxy gambogic acid or gambogic acid or a pharmaceutically acceptable derivative thereof, in addition to a therapeutically effective amount of one or more of cisplatin, cetuximab, gefitinib, erlotinib, afatinib, dacomitinib, osimertinib, rociletinib, omultinib, or doxorubicin. In certain embodiments, the treatment regimen can also include radiation (either photon or proton).

The term “pharmaceutically acceptable derivative” as used herein refers to and includes any pharmaceutically acceptable salt, pro-drug, metabolite, ester, ether, hydrate, polymorph, solvate, complex, and adduct of a compound described herein which, upon administration to a subject, is capable of providing (directly or indirectly) the active ingredient. For example, the term “a pharmaceutically acceptable derivative” of gambogic acid includes all derivatives of gambogic acid (such as salts, pro-drugs, metabolites, esters, ethers, hydrates, polymorphs, solvates, complexes, and adducts) which, upon administration to a subject, are capable of providing (directly or indirectly) gambogic acid. In some embodiments, the functional groups of a gambogic acid or a 30-hydroxygambogic acid is modified to alter certain biological effects, such as to improve potency, to decrease side effects, or to increase absorption.

As used herein, the term “pharmaceutically acceptable salt” refers to those salts, which retain the biological effectiveness and properties of gambogic acid or 30-hydroxygambogic acid. And unless otherwise indicated, a pharmaceutically acceptable salt includes salts of acidic or basic groups, which may be present in the compounds of the formulae disclosed herein. The present disclosure also provides certain processes, as examples, for the preparation of the above pharmaceutically acceptable salts, their derivatives, their analogs, their tautomeric forms, their stereoisomers, their polymorphs, and pharmaceutical compositions containing them.

Certain embodiments relate to pharmaceutically acceptable salts formed by gambogic acid or 30-hydroxygambogic acid, their derivatives, their analogs, their tautomeric forms, their stereoisomers, their polymorphs and pharmaceutically acceptable compositions containing them. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenylsubstituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

Embodiments of the invention include pharmaceutical compositions including gambogic acid or 30-hydroxy gambogic acid, or a pharmaceutically acceptable derivative, and a pharmaceutically acceptable carrier or excipient. Pharmaceutically acceptable ingredients, such as excipients, diluents, fillers, binders, and carriers can be inert or actively contribute to the delivery and distribution of gambogic acid or 30-hydroxy gambogic acid. The formulations used in embodiments herein include excipients, such as microcrystalline cellulose, lactose monohydrate, hydroxypropyl cellulose, croscarmellose sodium and magnesium stearate, preferably at least about 50 wt %, such as in the range from about 50% to about 95 wt %, including the range from about 50-90 wt %, and more preferably in the range from about 55-85 wt %, such as in the range from about 60% to about 85 wt %, or in the range from about 65 wt % to about 80 wt %, including about 60 wt %, about 65 wt %, about 70 wt %, about 75 wt %, or about 80 wt %.

As used herein, a “therapeutically effective amount” is an amount of an active ingredient (e.g., gambogic acid, 30-hydroxygambogic acid, cisplatin, cetuximab, gefitinib, erlotinib, afatinib, dacomitinib, osimertinib, rociletinib, omultinib, or doxorubicin) or an pharmaceutically acceptable salt thereof that eliminates, ameliorates, alleviates, or provides relief of the symptoms for which it is administered, and, as such, a “therapeutically effective amount” depends upon the context in which it is being applied. A therapeutically effective amount of a compound of gambogic acid and 30-hydroxygambogic acid can be administered in one or more administrations. As used herein, the terms “management,” “managing,” “manage,” “treatment,” “treating,” and “treat” refer to any indicia of success in the treatment or amelioration of an injury, disease, or condition, including any objective or subjective parameter such as abatement; remission; diminishing of symptoms or making the injury, disease, or condition more tolerable to the subject; slowing in the rate of degeneration or decline; making the final point of degeneration less debilitating; and/or improving a subject's physical or mental well-being.

Innovative and safer therapeutic strategies, such as targeted therapies, are needed to safely combat the growing HPV⁺-HNSCC epidemic. HPV oncoproteins, particularly E6, represent a unique and potentially therapeutically favorable strategic approach for targeted HPV⁺-HNSCC treatment. E6 is a causative agent in the cellular transformation and immortalization of keratinocytes, and its continuous expression is necessary to maintain tumor progression. E6 also modulates the survival of HPV⁺ tumor cells by impacting how they respond to apoptotic stimuli. This occurs primarily through inhibitory protein-protein interactions with proteins, such as p53 and caspase 8. E6 directly binds to proteins in the extrinsic apoptotic pathway, such as caspase 8. E6 physically binds to proteins of the intrinsic apoptosis pathway, such as p53 and Bak, and consequently facilitates their proteasomal degradation. Furthermore, such E6-mediated inhibition of caspase 8 blunts the induction of cell death of HPV⁺ cells by apoptosis-inducing cancer therapies. Absence of p53 and caspase 8 in HNSCC is correlated with attenuation of sensitivity of HPV⁺-HNSCC to chemotherapy and radiation. Consistent with this, genetic tools such as CRISPER, TALEN gene knockouts, RNAi, and other agents that indirectly knock down E6 mRNA have demonstrated that depleting the protein abundance of E6 leads to anti-proliferative effects and enhances the response of HPV⁺ cells to chemotherapy agents and radiation. Thus, E6 acts by blocking apoptosis, and its critical role as a survival factor in HPV⁺ tumors makes it an attractive therapeutic target.

Provided here are small molecule inhibitors that disrupt binding of E6 to caspase 8. The AlphaScreen technology™ (Perkin Elmer, Waltham, MA) was used as a preliminary screening strategy. This technology is a proximity-based platform for identifying hit compounds that perturb a specific interaction between two beaded proteins. Using this approach, a library of over 5000 small molecules was interrogated for compounds that antagonize E6 binding to caspase 8. Approximately 96 hits were identified, and further characterized through a number of complementary and orthogonal tests to authenticate their activity and specificity. GA-OH emerged as the most promising inhibitor of E6, and in follow-up cell-based studies, showed selective growth suppression and increased cell death. These results suggest strategies for the development of novel therapies for HPV⁺-HNSCC.

### Primary Screening and Hit Identification

Three structurally diverse libraries (Prestwick library, Microsource Spectrum library and an in-house collection at Kansas University; see Table 1) were screened for the ability of compounds to inhibit the binding of full-length HPV E6 to human Caspase 8 using a previously optimized AlphaScreen™ protocol.

TABLE 1
IC₅₀ (μM) values against E6-Caspase 8 binding for the 96 compounds that were selected as initial hits
following the primary screen. The 69 hits selected for counter-screening are highlighted as bold text.

| Compound ID | IC₅₀ (μM) | Compound ID | IC₅₀ (μM) | Compound ID | IC₅₀ (μM) | Compound ID | IC₅₀ (μM) |
|---|---|---|---|---|---|---|---|
| KU0101350 | 0.08 | KU0101639 | 1.71 | KU0102344 | 4.85 | KU0103230 | 2738.00 |
| KU0101521 | 0.30 | KU0103046 | 1.81 | KU0102544 | 4.92 | KU0105079 | 1.87E+28 |
| KU0101407 | 0.35 | KU0101640 | 2.07 | KU0102272 | 4.99 | KU0103392 | ~2.215e+008 |
| KU0101633 | 0.57 | KU0101863 | 2.09 | KU0102576 | 5.15 | KU0105025 | ~2.518e+007 |
| KU0101351 | 0.57 | KU0101505 | 2.26 | KU0102217 | 5.21 | KU0103558 | ~21210 |
| KU0101562 | 0.64 | KU0102051 | 2.32 | KU0102474 | 5.79 | KU0104995 | ~28789 |
| KU0101152 | 0.76 | KU0101513 | 2.45 | KU0102410 | 6.08 | KU0103640 | ~3.507e+006 |
| KU0101570 | 0.76 | KU0101869 | 2.45 | KU0102635 | 6.12 | KU0104819 | ~35931 |
| KU0101353 | 0.88 | KU0101837 | 2.46 | KU0102227 | 6.18 | KU0103575 | ~378185 |
| KU0101587 | 0.98 | KU0102558 | 2.75 | KU0102735 | 7.13 | KU0105084 | ~4.523e+008 |
| KU0101202 | 1.15 | KU0101845 | 2.76 | KU0102371 | 7.14 | KU0103589 | ~4.965e+013 |
| KU0101667 | 1.23 | KU0102486 | 2.78 | KU0102799 | 7.17 | KU0104990 | ~6.513e+007 |
| KU0101354 | 1.29 | KU0101853 | 2.89 | KU0102165 | 7.30 | KU0103518 | ~8170 |
| KU0101484 | 1.33 | KU0102566 | 3.03 | KU0102807 | 7.76 | KU0105263 | Ambiguous |
| KU0101228 | 1.35 | KU0101965 | 3.27 | KU0102510 | 8.90 | KU0103598 | Ambiguous |
| KU0101870 | 1.37 | KU0103086 | 3.48 | KU0102848 | 9.08 | KU0106020 | Ambiguous |
| KU0101357 | 1.43 | KU0102126 | 3.58 | KU0044882 | 9.15 | KU0104019 | Ambiguous |
| KU0101886 | 1.47 | KU0103094 | 3.59 | KU0104071 | 10.74 | KU0106101 | Ambiguous |
| KU0101558 | 1.49 | KU0102278 | 3.92 | KU0102753 | 10.74 | KU0103872 | Ambiguous |
| KU0101902 | 1.592 | KU0102431 | 4.189 | KU0103920 | 10.9 | KU0106021 | Ambiguous |
| KU0101566 | 1.618 | KU0102151 | 4.287 | KU0102777 | 10.92 | KU0103810 | Ambiguous |
| KU0101958 | 1.619 | KU0102551 | 4.358 | KU0104003 | 37.07 | KU0106102 | Ambiguous |
| KU0101598 | 1.62 | KU0103055 | 4.701 | KU0103302 | 37.55 | KU0104231 | Ambiguous |
| KU0101982 | 1.673 | KU0102512 | 4.843 | KU0104381 | 58.77 | KU0106103 | Ambiguous |

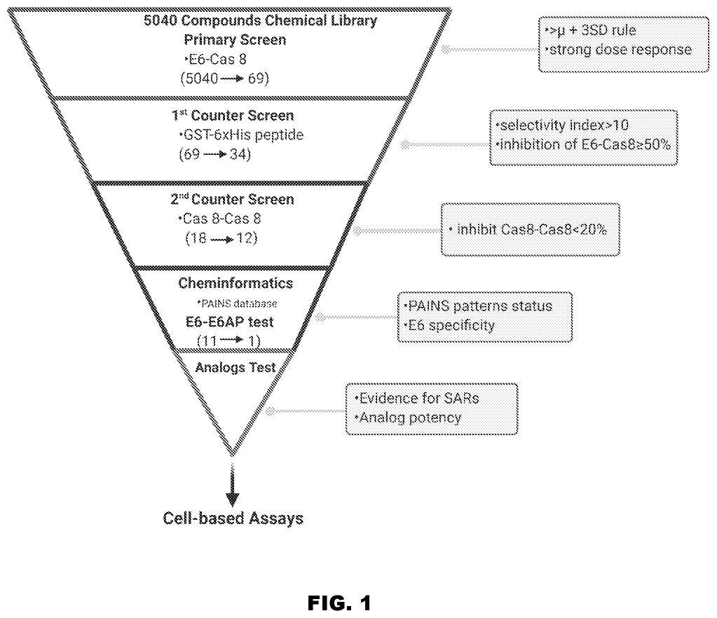

FIG. 1 is a diagrammatic representation of the high content screening strategy for certain embodiments. Screening funnel scheme shows the screening activity performed at each given step of hit compound triage and the respective decision criterion used for hit selection. Primary screening was followed by secondary screening and cheminformatics filtering using PAINS databases. Relationships between structure and activity were then analyzed before one candidate was chosen for cell studies. For each of the compounds screened, % inhibition was calculated. A histogram plot of all the compounds against their % inhibition displayed a normal distribution.

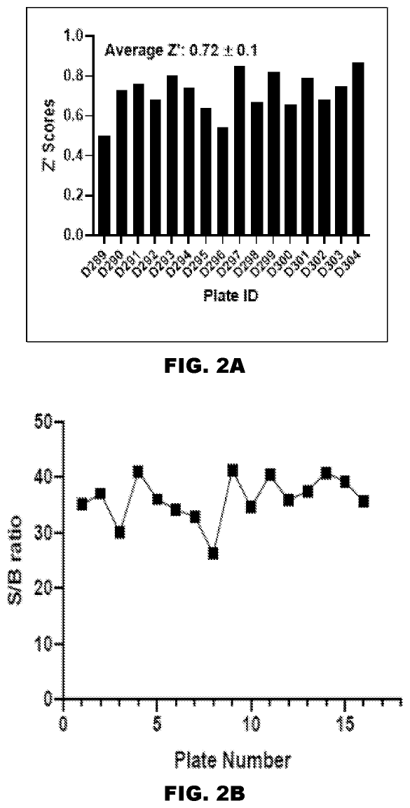

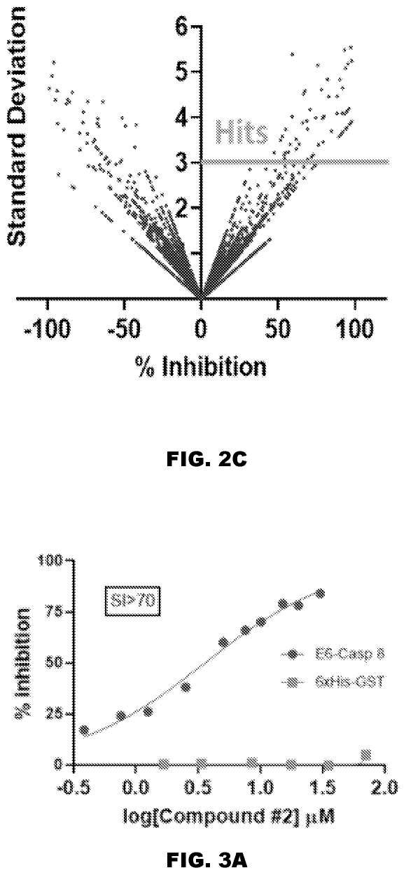

Three variables for assay optimization—Z-factor (a measure of statistical effect size), signal to background ratio, and the coefficient of variation—were evaluated for the assay quality and performance. The resulting statistical parameters from the screening data indicated good statistical validation and adequate suitability of the assay for high content screening. FIGS. 2A-2C are graphical representations of the Z-factor, signal to background ratio (S/B), and the coefficient of variation (CV %). FIG. 2A is a graphical representation of Z factor scores were determined for each plate, and the median and average scores were found to both be >0.6, indicating suitability of the assay for high content screening. FIG. 2B is a graphical representation of the signal to background ratio for each plate. FIG. 2C is a scatter plot compound (compounds above the solid green line). Hits were selected using a computed cut off value of % Inhibition 3SD rule. Z-factor>0.5 are considered the threshold for the assay to be considered excellent and suitable for high content screening. The median and mean Z-factors that were calculated for the sixteen 384-well plates used to screen the 5k library were 0.72 and 0.67, respectively. These Z-factor scores demonstrate suitability for high throughput screening. Similarly, the assay also demonstrated high sensitivity with the mean S/B ratio of 36 (FIG. 2B). Variability between plates was also low for all the 16 plates with the mean CV of 9.6%, and well below the acceptable threshold of <20%. With these quality control parameters well within the acceptable ranges and suggesting overall robustness, the focus was turned on identifying possible hit compounds. Standard deviation from sample mean for each compound was plotted against % inhibition of E6 binding to caspase 8. The μ+3SD rule was applied to the normalized % inhibition data, and compounds that were 3 Z-scores above the sample average were selected (FIG. 2C). With this hit selection cutoff, 96 compounds were selected as preliminary hits for an initial hit rate of about 1.9%. These 96 compounds were then subjected to dose-response analysis to assess competitive behavior as well as the relationship between concentration and inhibitory activity on E6 binding. Of the initial hits, 69 displayed a strong dose response as demonstrated by clear sigmoidal behavior and IC₅₀ values of 10 μM or lower (Table 1), and were thus selected for secondary screening as discussed below.

### Counter-Screening and Hit Confirmation

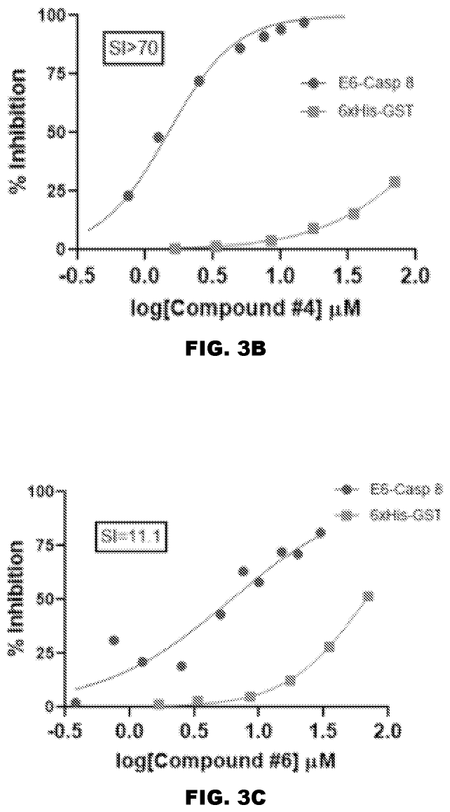

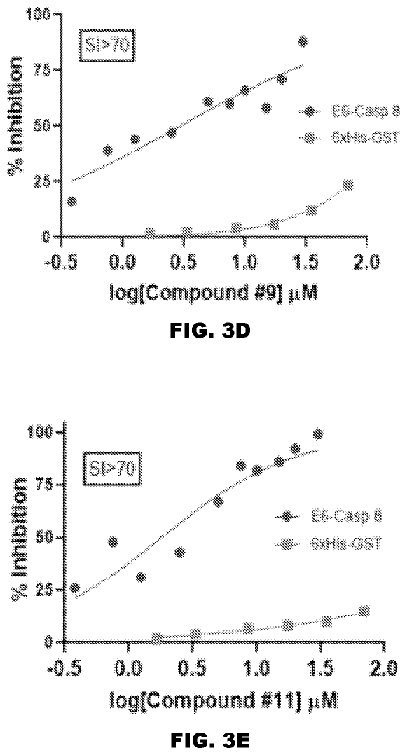

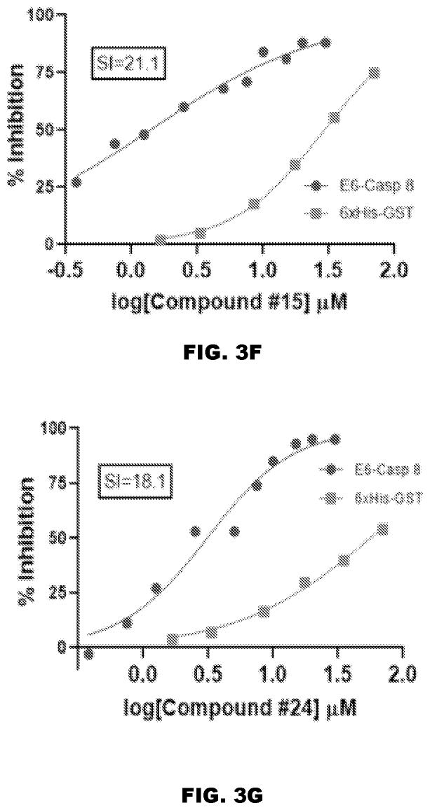

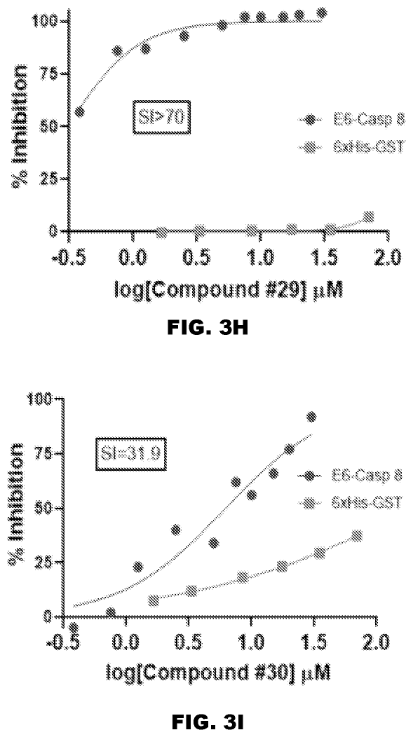

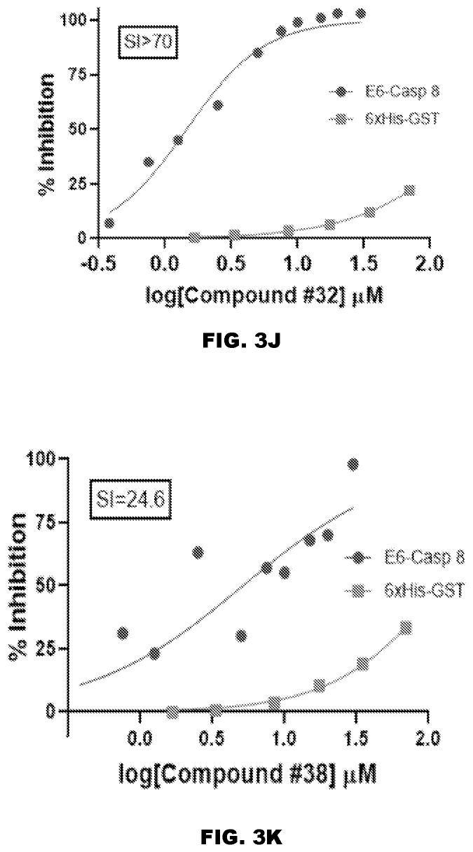

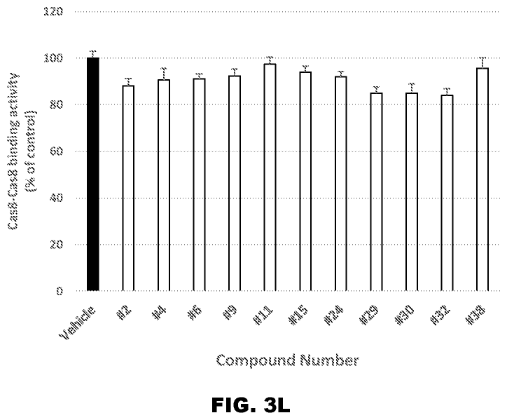

In the AlphaScreen™ method, artifacts that interfere with aspects of signal generation and bead capture, rather than the binding of the two proteins being assayed, may initially be identified as hits. A counter-screen is necessary to eliminate compounds with such non-specific and promiscuous interactions. To do this, two distinct counter-screens were employed. For the first, the GST-6xHis fusion peptide was utilized. This peptide, containing the affinity handles of E6 and caspase 8 respectively, represented the null control reaction. To assess specificity, the primary screen (E6-Caspase 8) was also performed in parallel. From the null and primary reactions, the selectivity index (SI) was calculated, and compounds with preferential inhibition of E6-Caspase 8 relative to GST-His₆ were chosen; the rest were removed from consideration as promiscuous. Thirty-four of the initial hit compounds displayed an SI>10; that is, about 50% of the initial hits were at least 10-fold more selective in inhibiting E6-Caspase 8 binding versus the control substrate. Conversely, about half of the compounds were identified as frequent hitters and thus non-selective. From these remaining hits, 18 compounds were selected based on commercial availability and strength of selectivity index, as well as whether their maximum inhibition of E6-Caspase binding was ≥50%. These compounds were then subjected to the second counter-screen. This counter-screen assessed the ability of compounds to interfere with GST-Caspase 8-His₆-Caspase 8 binding, rather than GST-E6-His₆-Caspase 8 binding, and its objective was to flag compounds that preferentially bind to caspase 8 rather than to E6, potentially interfering with host cell apoptosis. The inclusion criteria for the preferred compounds in this screen was set to less than 20% inhibition of Caspase 8-Caspase 8 binding. Using this criterion, 11 of the 18 compounds were taken as “true” primary hits for a confirmed hit rate of 0.22%. The selectivity profiles and indices of the 11 compounds are shown in FIG. 3. The IC₅₀ values of these compounds against E6-caspase 8 binding is also shown in Table 2.

TABLE 2
IC₅₀ (μM) values against E6-Caspase 8 binding
for the 11 compounds that passed counter-screening.

| Hit Compound No. | IC₅₀ (μM) |
|---|---|
| #2 | 3.59 |
| #4 | 1.48 |
| #6 | 6.06 |
| #9 | 2.94 |
| #11 | 1.80 |
| #15 | 1.39 |
| #24 | 2.00 |
| #29 | 0.61 |
| #30 | 6.23 |
| #32 | 1.46 |
| #38 | 5.17 |

FIGS. 3A-3K are graphical representations of the selectivity profiles and indices of the selected 11 compounds. Binding activity graphs for the 11 compounds that passed both the 6xHis-GST and Caspase 8-Caspase 8 counter-screens are shown. The graphs show activity of each compound against Caspase 8-E6 and 6xHis-GST binding, and the calculated SI values from the activity against these two types of substrates (IC₅₀/IC₅₀) are shown.

The binding profiles show that these compounds exhibit little to no interference with the assay itself. FIG. 3L is a graphical representation of the interaction of these compounds with the dimerization of caspase 8. In addition, these compounds also show little interaction with the dimerization of caspase 8 as shown in FIG. 3L. These results indicate that they are specific inhibitors of the interaction between E6 and caspase 8.

### Cheminformatic Analysis

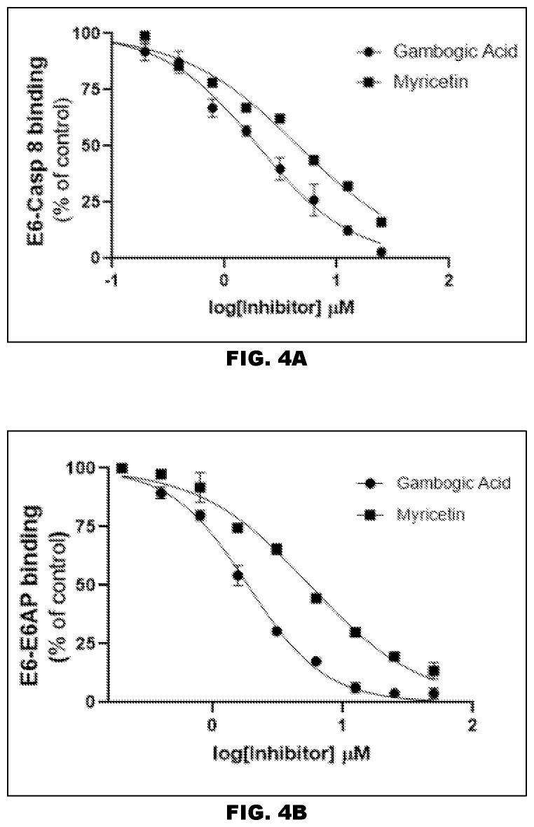

To prioritize the remaining 11 hit compounds for downstream analysis such as SAR and cell-based functional studies, a more qualitative cheminformatic approach was undertaken. Our goal was to prioritize compounds with no known promiscuity in biochemical assays by looking for the presence of PAINS (Pan-assay interference compounds) patterns. Using four PAINS-detector online tools that recognize substructures of frequent hitter compounds, any flagged compound was excluded and only those compounds were selected that came out as PAINS-free in all four runs. This analysis was complemented by an examination of the remaining compounds to flag bad functional groups (BFGs) or problematic substructures that could have been missed computationally. Comparison with literature findings further enabled us to keep only those compounds that possessed novel activity against E6. After these steps, gambogic acid (compound #24) remained the best candidate for further studies. Its activity was then cross-validated by performing additional AlphaScreening™ tests using GST-E6 and His₆-E6AP as substrates. Myricetin, a known E6 inhibitor that prevents binding of both caspase 8 and E6AP to E6, was included as a positive control in this assay. The activity of these two inhibitors against E6-Caspase 8 binding were evaluated in parallel for a head to head comparison. Compared to myricetin, gambogic acid displayed greater potency than myricetin against binding to both substrates with inhibitory concentrations that were at least two-fold lower (IC₅₀ 1.9 μM vs. 4.6 μM against E6-Caspase 8 and IC₅₀ 1.7 μM vs. 5.6 μM against E6-E6AP). FIGS. 4A-4B are graphical representations of the activity of gambogic acid as compared to myricetin in binding assays with caspase 8 and p53, respectively. FIG. 4A is a graphical representation of the head-to-head comparison of gambogic acid with myricetin with respect to activity against E6 binding to Caspase 8. FIG. 4B is a graphical representation of the head-to-head comparison of gambogic acid with myricetin with respect to activity against E6 binding to E6AP. The inhibitory activity of gambogic acid as measured by AlphaScreen™ is superior to that of myricetin. These findings suggest potential for rescuing caspase 8 and p53 functions in cells.

### SAR Analysis

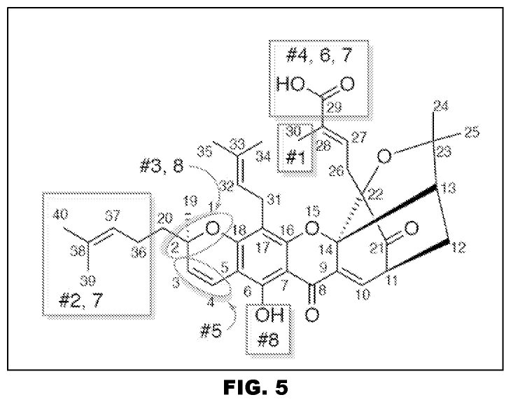

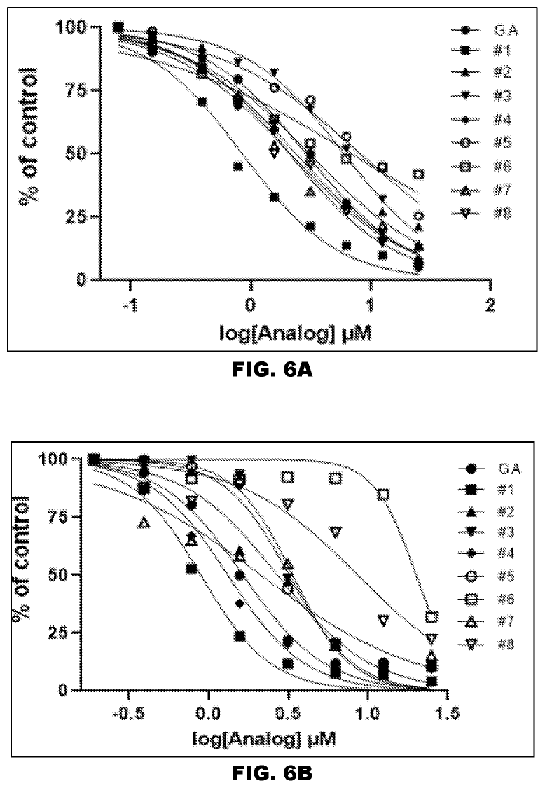

Guided by this information, eight analogs of gambogic acid were purchased from commercial vendors to carry out a limited structure-activity relationship (SAR) analysis (FIGS. 5 and 6). FIG. 5 is an image showing the analogs of gambogic acid following structure-activity relationship analysis. Rings A, B, C, D make up the core scaffold of gambogic acid. Analog #1 is 30-hydroxygambogic acid (GA-OH), analog #2 is morellic acid, analog #3 is gambogenic acid, analog #4 is gambogic amide, analog #5 is neo-gambogic acid, analog #6 is gambogin, analog #7 is iso morellinol, and analog #8 is acetyl iso-gambogic acid.

The AlphaScreen™ analysis of these structural analogs were performed for their ability to inhibit E6-caspase 8 binding. Generally, the inhibitory activity amongst the analogs were similar due to their structural similarity (FIGS. 5 and 6). FIGS. 6A-6B are graphical representations of the activity profiles of analogs of gambogic acid in vitro (AlphaScreen) and in vivo (HPV⁺ cell line assays). FIG. 6A is a graphical representation of the E6 hit analog activity in vitro using E6-Caspase 8 substrates. With the exception of #3, 5, and 6, most analogs show activity close to that of the parent compound. FIG. 6B is a graphical representation of the E6 hit analog activity in vivo (cells) context, #3, 5, and 6 also show low activity; #8 is a surprise addition to this group. #1 remains the most promising lead in terms of activity in the HPV⁺ cell line. That said, the activity of three of these analogs (#3, 5, and 6) deviated noticeably (˜>3 logs less active) from the parent compound. Analogs #3 and 5 had modifications to ring A from the core scaffold of gambogic acid. Analog #3 had the A ring cleaved, while analog #5 had the C3/4 olefin oxidized and a hydroxyl group at C4. The modification on analog #6 was distant from the core ring system, with the compound lacking the carboxylic acid at C29, possessing instead an unoxidized methyl group. The structural changes represented by analogs #2 (removal of the isoprenyl group at C2), #4 (replacement of the carboxylic acid at C29 with a primary amide), #7 (removal of the isoprenyl at C2 and reduction of the C29 carboxylic acid to an alcohol) and #8 (acetylation of the C8 phenol and epimerization at C2) did not significantly affect the activity of the analogs relative to GA. Notably, analog #1, 30-hydroxygambogic acid, more effectively inhibited E6 binding to caspase 8 than any of the other analogs or the parent compound. The increased binding of analog #1, possessing a hydrogen-bonding hydroxyl group at C30, and the loss of activity of analog #6, which removed the carboxylic acid at C29, indicate the importance of the oxidized isoprenyl group at C22 to the activity of these compounds against E6. The unaffected activity of C29 amide analog #4 is also significant.

Next, the analogs were evaluated for functional activity using the HPV⁺ HNSCC cell line, SCC104, in the MTT assay as described previously to determine whether similar findings would be observed as in the in vitro AlphaScreen™ analysis. With one exception, the patterns were similar to the AlphaScreen™ data, but the differences were relatively more pronounced in the cell-based screen (FIG. 6B). Analogs #3, 5 and 6 had the third, fourth, and first highest IC₅₀ₛ relative to the parent compound, respectively. The only compound that significantly differs from the trend seen in the AlphaScreen™ results is analog #8, which showed high activity in vitro but diminished activity in this cell-based assay. As in the AlphaScreen™ analysis, analog #1 (30-hydroxygambogic acid) showed the highest potency of all analogs, including the parent compound. Based on these findings, 30-hydroxygambogic acid (GA-OH) was selected as a candidate for more extensive functional studies.

### GA-OH Selectively Inhibits Cell Growth and Cell Survival in HPV⁺ Cell Lines

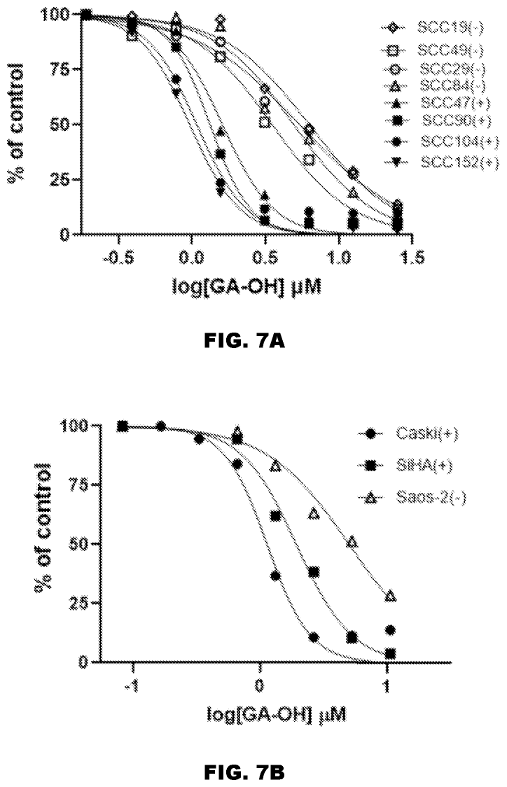

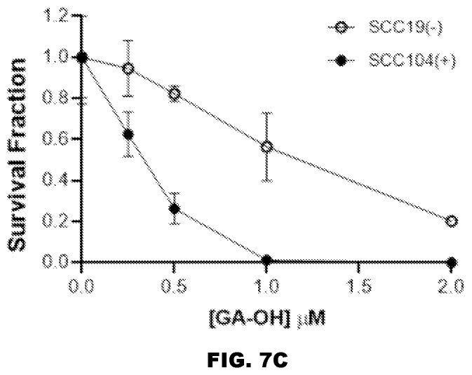

While SAR studies using the SCC104 cell line provided evidence of the activity of the analogs, further studies were conducted to assess not just the potency, but also the selectivity. As a step towards that goal, the efficacy of GA-OH was evaluated in a panel containing both HPV⁺ and HPV⁻ HNSCC cell lines using MTT cell viability assays. Four HPV⁺ cell lines (SCC47, SCC090, SCC104, SCC152) and four HPV⁻ cell lines (SCC19, SCC29, SCC49, SCC84) were utilized. GA-OH behaved dose dependently in cell lines both with or without HPV. However, the HPV⁺ cell lines tested here displayed higher sensitivity than did the HPV⁻ cell lines (FIG. 7A). FIGS. 7A-7C are graphical representations of the sensitivity of HPV⁺ cell lines and HPV⁻ cell lines to GA-OH-mediated growth inhibition. FIG. 7A is a graphical representation of the cell growth inhibition of HNSCC HPV⁺ and HPV⁻ cell lines by GA-OH (+ and closed shapes represent HPV⁺ cell lines and − and open shapes the HPV⁻ cell lines). These differentials in activity between HPV⁺ and HPV⁻ cell lines were consistent with AlphaScreen™ results that showed evidence that GA-OH could inhibit E6 interactions with pro-apoptotic molecules such as caspase 8 and E6AP. To further validate these results, the activity of GA-OH was also tested in a panel of cervical cancer (CC) cell lines. These cells were good controls, as HPV is an established causative agent in CC carcinogenesis. Two HPV⁺ cell lines, SiHa and CaSki were selected as the positive controls and the Saos-2 cell line was used as an HPV-negative control. A similar pattern in selectivity differentiated by HPV status of the cell line was also observed (FIG. 7B). FIG. 7B is a graphical representation of the cell growth inhibition of cervical cancer HPV⁺ and HPV⁻ cell lines by GA-OH. HPV⁺ cell lines display higher sensitivity than do HPV⁻ cell lines as shown by the leftwards shift of the HPV⁺ curves (SCC47, 90, 104, 152 in FIG. 7A and SiHa and CaSki in FIG. 7B).

For assessing the long-term effects on the survival of cells following GA-OH treatment, the colony formation assay (CFA) was performed. Two cell lines, SCC19 (HPV⁻) and SCC104 (HPV⁺) were used for this study. The cells were treated for 24 hours with GA-OH, and then seeded for assessment of colony formation. Cell survival data from this study mirrored the impact of GA-OH on cell viability of HPV⁺ and HPV⁻ cell lines. The number of colonies in the HPV⁺ cell line was significantly and dose-dependently reduced at every dose of GA-OH tested compared to the SCC19 cell line. On the other hand, the number of colonies in SCC19 did not exhibit significant reduction relative to their control except at high concentrations of GA-OH (FIG. 7C). FIG. 7C is a graphical representation of the effects of GA-OH on the clonogenicity of the surviving fractions of HPV⁺ versus HPV⁻ cell lines. GA-OH displayed higher cytotoxicity to HPV⁺ cells. This shows that GA-OH treatment has long-lasting effects on the viability and subsequent survival of HPV⁺ cells as compared to HPV⁻ cells.

### GA-OH Stabilizes p53 Levels and Induces Apoptosis in HPV⁺ Cells

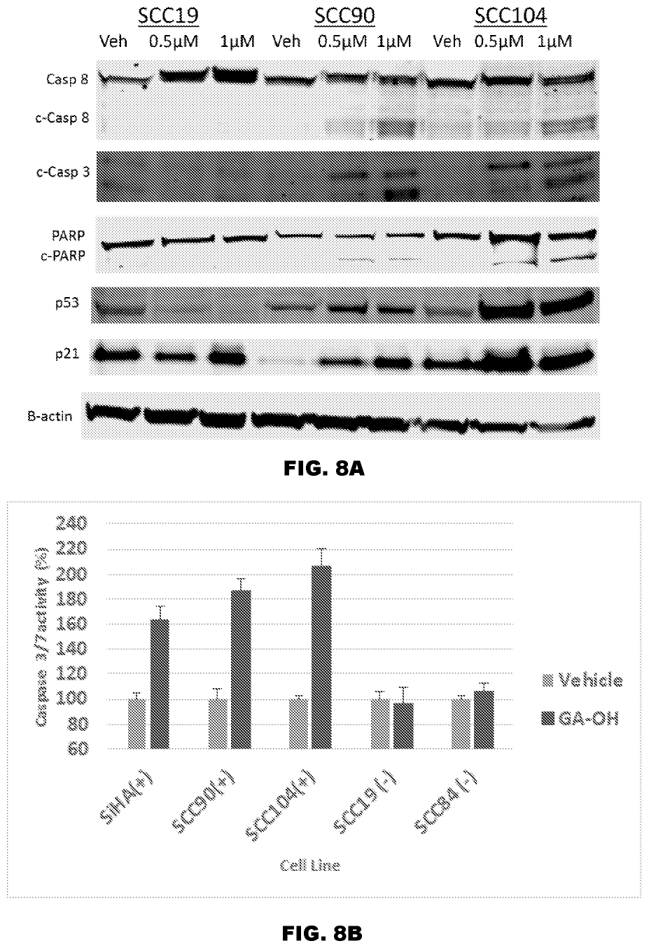

Based on the cell viability studies described above, as well as AlphaScreen™ data showing that that GA-OH prevents E6 from binding to both caspase 8 and the p53-recruiter, E6AP, activation of p53 and associated apoptotic effects could be contributing to the decrease in cell viability. Levels of p53 and its target gene product, p21, were evaluated using immunoblotting. Treatment with GA-OH resulted in an increase of p53 in both the HPV⁺ SCC90 and SCC104 cell lines, but not their HPV⁻ counterpart (SCC19) (FIG. 8A). FIG. 8A is a photographic image of blots analyzing Caspase 8 expression and activation of downstream targets. SCC19, SCC90 and SCC104 were seeded and treated with vehicle, 0.5 μM and 1 μM Of GA-OH for 24 hours. Activation of p53 was tested by blotting for p53 expression and activation. Caspase 8 activation and apoptosis was tested by blotting for Caspase 8 expression and activation of downstream targets. These observations were corroborated by an induction of the levels of the target of p53, p21. The data shows a robust induction of p21 levels compared to the vehicle control. The basal levels of p53 in the HPV⁺ cell lines are lower compared to the HPV⁻ cell line.

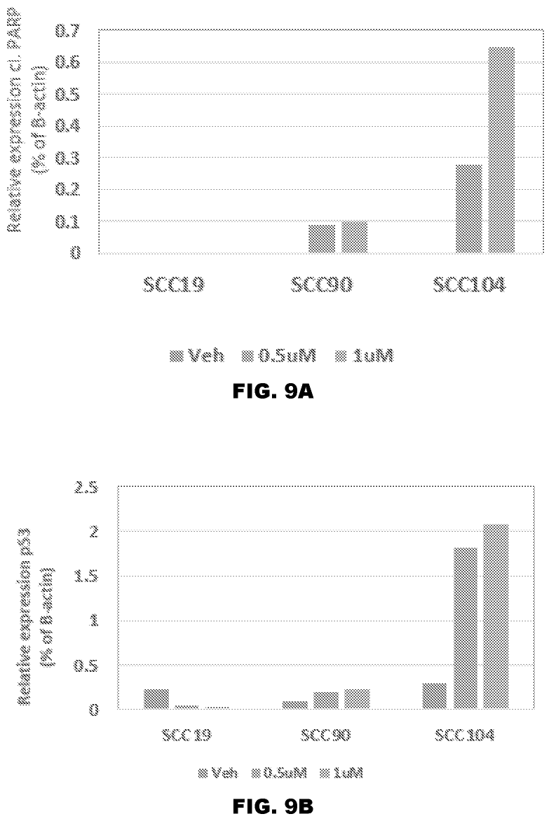

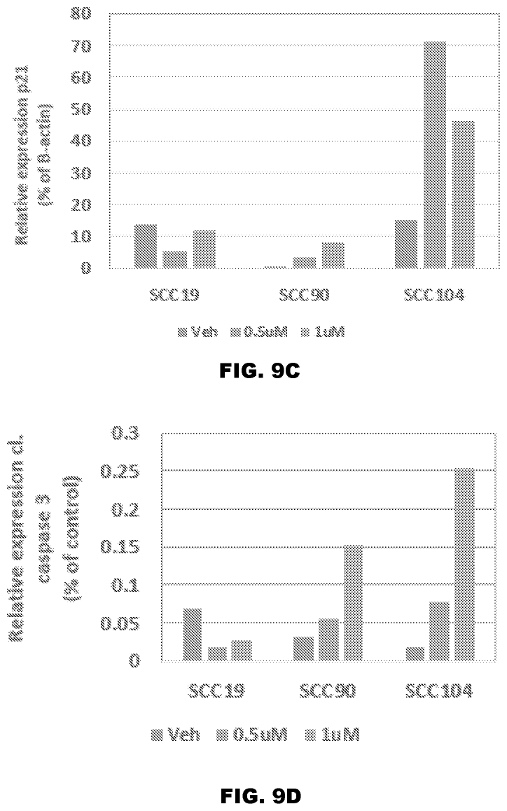

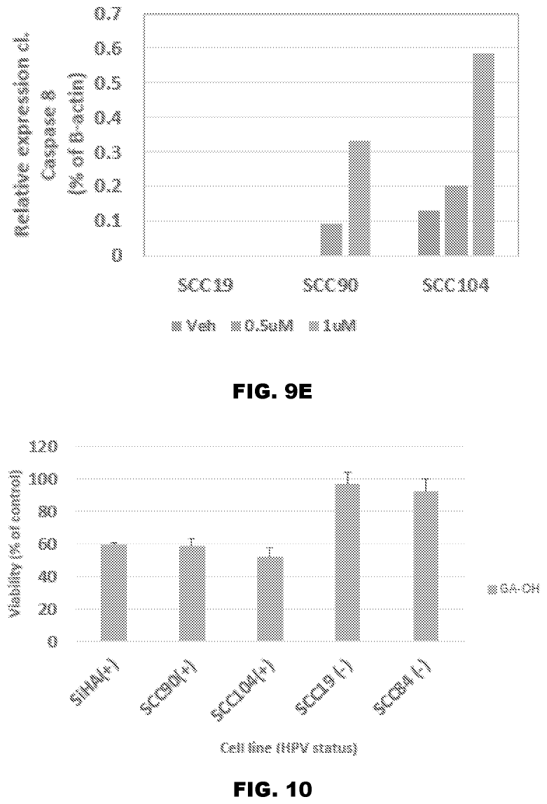

The effect of GA-OH treatment on activation of another target of E6, caspase 8, and its downstream targets in the apoptosis cascade was evaluated. Western blot analysis shows that GA-OH treatment leads to cleavage of caspase 8 in a dose-dependent manner. There is a noticeable increase in caspase 8 levels in the HPV⁻ cell line upon exposure to GA-OH. However, there is no visible cleavage of caspase 8 itself (FIG. 8A). A look at the down-stream effectors of apoptosis shows a similar trend. Caspase 3 is cleaved dose-dependently, as was observed with caspase 8. PARP is also cleaved, even though its cleavage is modest. Quantification of relative expression of each of the targets above using B-actin as a loading control. FIGS. 9A-9E are graphical representations of the relative expression of cleaved PARP, p53, p21, cleaved caspase 3, and cleaved caspase 8, respectively in SCC19, SCC90 and SCC104 cell lines when treated with vehicle, or 0.5 μM, or 1 μM of GA-OH, each of the targets above using B-actin as a loading control is shown.

These findings were then confirmed by conducting a Caspase 3/7 activity Glo assay, which is a surrogate for apoptotic induction. Three HPV⁺ cell lines (SCC090, SCC104, SiHa) and two HPV⁻ cell lines (SCC19, SCC84) were assessed for activity of caspases 3 and 7, and thus for apoptosis activity. Significant apoptosis induction was observed in HPV⁺ cell lines compared to the controls (FIG. 8B). FIG. 8B is a graphical representation of the Caspase 3/7 activity of gambogic acid in various HPV+ and HPV− cell lines. These cell lines were seeded in 96 well plates and treated with 0.75 uM GA-OH. Caspase 3/7 activity was measured after 24 hours. HPV⁺ cell lines show high levels of Caspase 3/7 activity. Cell viability was also assessed in parallel to corroborate this result. Cells with higher apoptosis induction generally also showed higher reduction in cell viability as measured by MTT. FIG. 10 is a graphical representation of the cell viability as measured by MTT, when three HPV⁺ cell lines (SCC090, SCC104, SiHa) and two HPV⁻ cell lines (SCC19, SCC84) were treated with gambogic acid. These results are consistent with the western blotting analysis involving caspase 8 and caspase 3, and also align with the AlphaScreen™ data showing that GA-OH inhibits E6 binding to caspase 8. Also, as seen by immunoblotting, little to no apoptosis activity is observed in the caspase Glo experiment for the HPV⁻ cell lines. Collectively, these results indicate higher cell viability suppression by GA-OH in HPV⁺ versus HPV⁻ cell lines.

Embodiments of this disclosure include new and novel inhibitors of the HPV oncoprotein E6 that have greater potential for therapeutic development. All compounds were screened using the AlphaScreen™ protocol. A number of filtration steps and gates consistent with field standard practices were embedded to make the hit identification process appropriately rigorous. Initial hit selection was based on criteria that a number of studies in the field have relied on, such as the statistically significant 3 Z-scores above the sample mean limit. Moreover, hits that met this criterion were excluded if they did not exhibit at least ˜50% inhibition of E6 binding. The primary hits were then subjected to secondary assays for further filtration. In counter-screening, hits with a selectivity index of at least 10 were chose; this minimum threshold is generally regarded as a rigorous starting point for choosing compounds demonstrating specificity.

The efficacy of GA-OH was further demonstrated in a biological context through cell-based assays. The results show that GA-OH suppressed cell proliferation and killed cells in an HPV-dependent manner, consistent with the role of E6 in cell growth and inhibition of apoptosis induction. In addition, activation of mediators of apoptosis, including p53 were also observed, particularly in the HPV⁺ cell models.

Gambogic acid showed activity against HPV⁻ cell lines, even though HPV⁺ cell lines were significantly more sensitive. Importantly, GA-OH was more potent and selective for HPV⁺ versus HPV⁻ cells as compared to gambogic acid, indicating that it has more specificity than its parent compound. GA-OH has improved solubility and the additional hydroxyl group appears to contributes to overall activity by providing another handle for hydrogen bonding between the molecule and E6. In addition, the extra polar group strengthens the hydrogen bond network that has been observed between small molecules and E6. In terms of the safety profile, it is highly unlikely that the additional hydroxyl group will decrease the tolerability of GA-OH relative to GA. GA has been found to be relatively tolerable in animal studies, and toxicities to organs were only observed at high concentrations.

One of the biggest and current unmet clinical needs for patients with HPV-associated HNSCC, particularly oropharyngeal cancer, is safety following treatment. The majority of patients present with locally advanced HNSCC and the standard of care consists of surgery with adjuvant therapy or chemoradiation. These standard treatments, which were originally designed for the more aggressive HPV-unrelated HNSCC, are intensive and cause severe long-term, treatment-related sequalae. However, it is now clear that HPV status is associated with not only greater response rates across all modalities but also better locoregional control and survival. Therefore, there is a possibility that the current treatment regimens of chemoradiation can be de-intensified to achieve similar survival outcomes and better functional outcomes and quality of life. This generally can be achieved by reducing the dosages and volumes of radiation or replacing cisplatin, the standard radiosensitizer. Currently, attempts to replace cisplatin have been done using cetuximab (an anti-EGFR monoclonal antibody), which is usually reserved for patients who do not tolerate the toxicity of cisplatin well. Unfortunately, early results from de-escalation clinical trials show that cetuximab is inferior to cisplatin in combination with radiation and is therefore not recommended for definitive therapy. Besides cetuximab, there are no other clinically approved targeted therapies for use in treating HNSCC that can be tested for de-escalation purposes. This shows that there is a great need for novel and selective agents that can act as radiosensitizers and be safely integrated into de-escalation regimens. To this end, we have tested our newly discovered E6-specific inhibitor in combination with radiation. Inhibition of E6 releases the brakes on apoptosis and will in principle enhance radiation-induced cell death synergistically through greater apoptotic induction. Preliminary findings indicate that GA-OH improves the effect of photon radiation in HPV⁺ cells but not HPV⁻ cells. Specifically, combination indices below were obtained for the two HPV⁺ cell lines used as summarized in Table 4 and these indices indicate synergistic effects according to the Bliss independence model. For HPV⁻ cell lines, no synergistic effects were observed when radiation was combined with GA-OH (see Table 4). GA-OH additively or synergistically interacts with cetuximab and cisplatin in HPV⁺ cells. In HPV⁻ negative cells, the interactions were antagonistic. The results of these interactions are also summarized in the table below. In certain embodiments, the additive or synergistic interactions of GA and GA-OH would extend to other EGFR-targeted agents, such as gefitinib, erlotinib, afatinib, dacomitinib, osimertinib, rociletinib, and omultinib.

Table 4A. Combination of GA-OH with different standard treatment agents. For GA-OH plus Cetuximab or Cisplatin an equal potency combination schedule was used to mix the inhibitors at potencies that reduce cell viability by 20% for each individual inhibitor (IC₂₀). For radiation, synergistic effects at radiation dose of 2Gy in combination with GA-OH are shown Combination indices for all the combinatorial treatments are shown. In the key below for degree of synergism/additivity/antagonism observed. CI represents combination index.

TABLE 4

| GA-OH plus Cetuximab | | | GA-OH plus Cisplatin | | | GA-OH plus Photon Radiation | | |
|---|---|---|---|---|---|---|---|---|
| Cell Line | CI | Symbol | Cell Line | CI | Symbol | Cell Line | CI | Symbol |
| SCC19(−) | 1.14 | − | SCC19(−) | 1.3 | −− | SCC19(−) | 1.16 | − |
| SCC29(−) | 1.19 | − | SCC29(−) | 1.2 | − | SCC29(−) | 1.04 | ± |
| SCC47(+) | 0.86 | + | SCC47(+) | 0.90 | + | SCC47(+) | 0.69 | +++ |
| SCC104(+) | 0.94 | ± | SCC104(+) | 0.97 | ± | SCC104(+) | 0.77 | ++ |
| SCC152(+) | 0.65 | +++ | SCC152(+) | 0.67 | +++ | SCC152(+) | N/A | N/A |

| Range of CI | Symbol | Description |
|---|---|---|
| <0.1 | +++++ | Very Strong Synergism |
| 0.1-0.3 | ++++ | Strong Synergism |
| 0.3-0.7 | +++ | Synergism |
| 0.7-0.85 | ++ | Moderate Synergism |
| 0.85-0.9 | + | Slight Synergism |
| 0.9-1.1 | ± | Nearly Additive |
| 1.1-1.2 | − | Slight Antagonism |
| 1.2-1.45 | −− | Moderate Antagonism |
| 1.45-3.3 | −−− | Antagonism |
| 3.3-10 | −−−− | Strong Antagonism |
| >10 | −−−−− | Very Strong Antagonism |

### GA-OH Sensitizes Cells to Radiation

CCDP, Cetuximab, and GA-OH were evaluated for their ability to sensitize HNSCC cell lines to photon radiation. Cells were treated with radiation using the given doses (0-4Gy) and treated with one of CDDP, cetuximab or GA-OH (Table 5 provides for concentrations of the various inhibitors).

TABLE 5
Concentrations of CDDP, Cetuximab, and GA-OH that were
used in combination with radiation for each cell line.

| CCDP + Photon | | GA-OH + Photon | | Cetuximab + Photon | |
|---|---|---|---|---|---|
| Radiation | | Radiation | | Radiation | |
| Cell Line | CCDP (μM) | Cell Line | GA-OH (μM) | Cell Line | Cetuximab (ng/mL) |
| SCC19(−) | 0.1 | SCC19(−) | 0.25 | SCC19(−) | 6 |
| SCC29(−) | 0.25 | SCC29(−) | 0.25 | SCC29(−) | 6 |
| SCC47(+) | 0.25 | SCC47(+) | 0.125 | SCC47(+) | 2 |
| SCC104(+) | 0.1 | SCC104(+) | 0.125 | SCC104(+) | 5 |
| SCC152(+) | 0.1 | SCC152(+) | 0.125 | SCC152(+) | 6 |

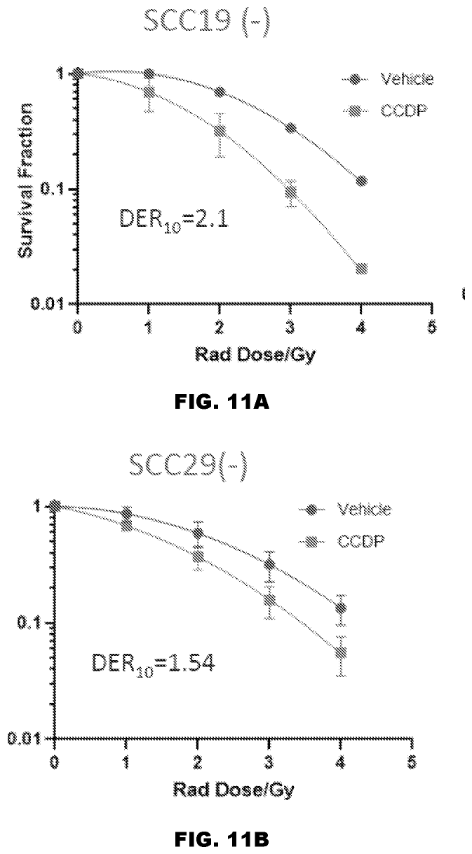

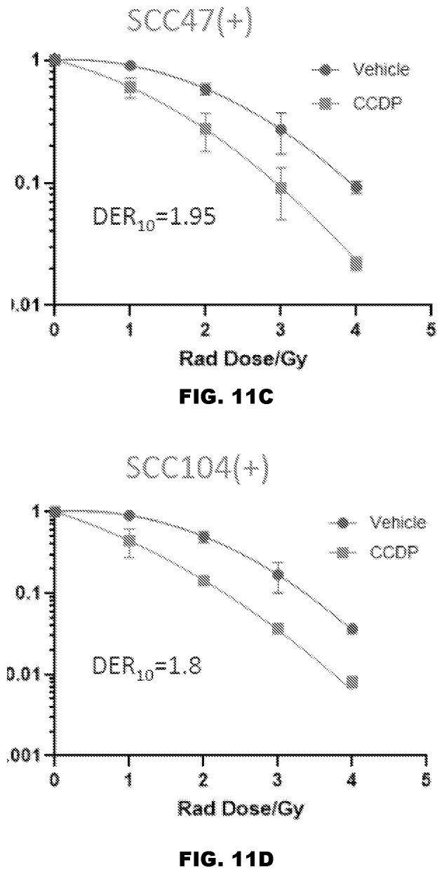

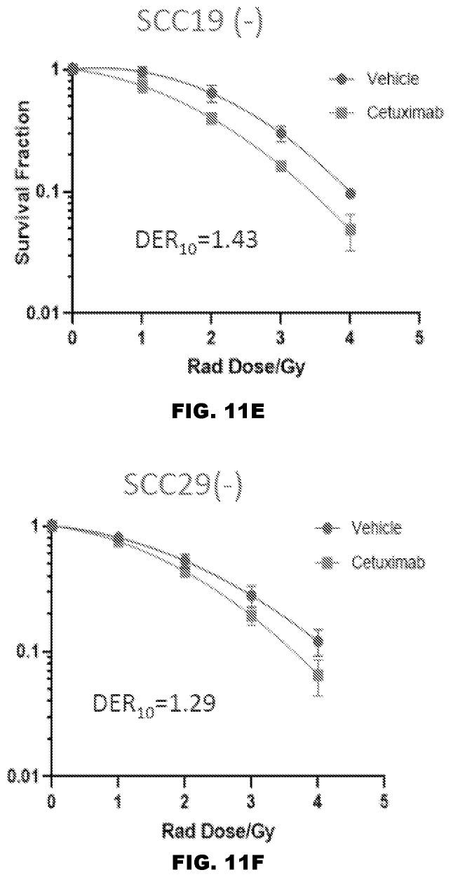

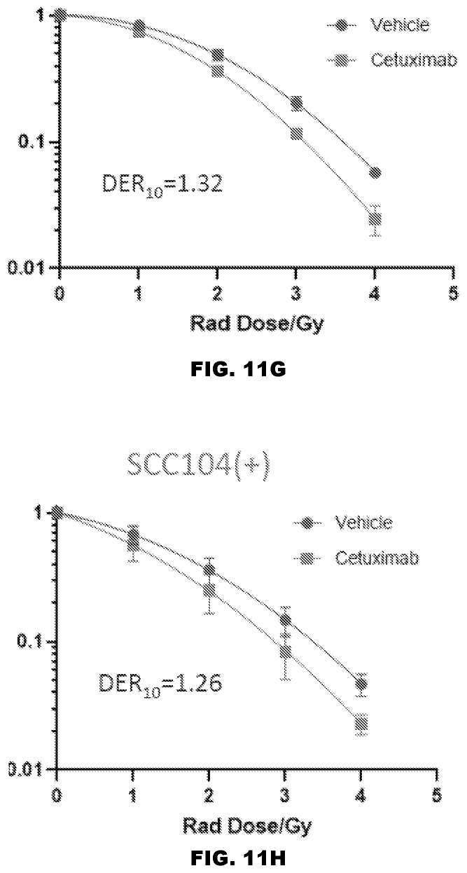

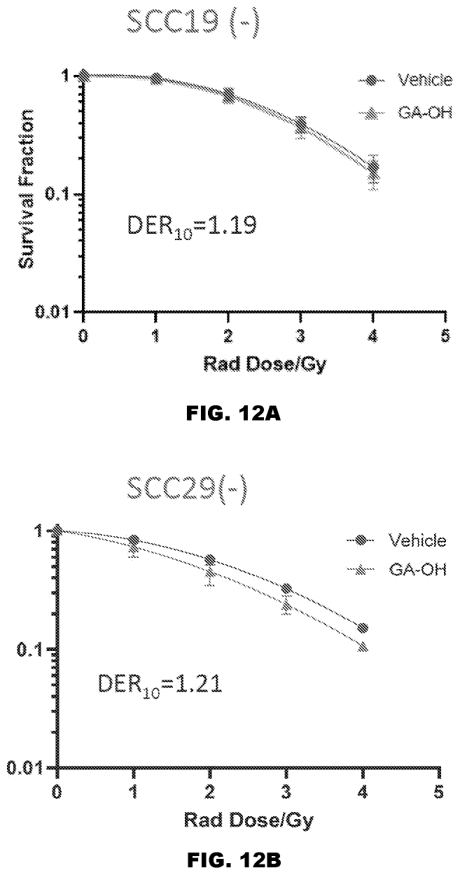

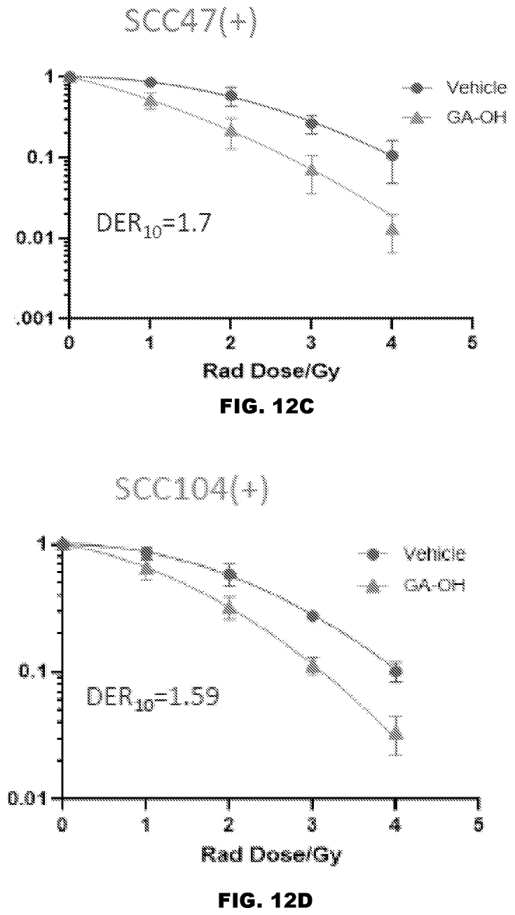

Colonies were counted after 14-21 days and normalized to vehicle control and survival analysis was performed. FIGS. 11A-11D are graphical representations of the radio-response of HPV⁻ cell lines (SCC19 and SCC29) and HPV⁺ cell lines (SCC47 and SCC104) when subject to photon radiation followed by treatment with either the vehicle alone or cisplatin (CCDP), as measured by MTT assays. Combination of CDDP and radiation shows sensitization in all cell lines regardless of HPV status. Cisplatin shows the greatest sensitization. FIGS. 11E-12H are graphical representations of the radio-response of HPV⁻ cell lines (SCC19 and SCC29) and HPV⁺ cell lines (SCC47 and SCC104) when subject to photon radiation followed by treatment with either the vehicle alone or cetuximab, as measured by MTT assays. Cetuximab also shows sensitization independent of HPV status. It shows the least sensitization. FIGS. 12A-12D are graphical representations of the radio-response of HPV⁻ cell lines ((SCC19 and SCC29) and HPV⁺ cell lines (SCC47 and SCC104) when subject to photon radiation followed by treatment with either the vehicle alone or GA-OH, as measured by MTT assays. GA-OH shows most sensitization in HPV+ cell lines.

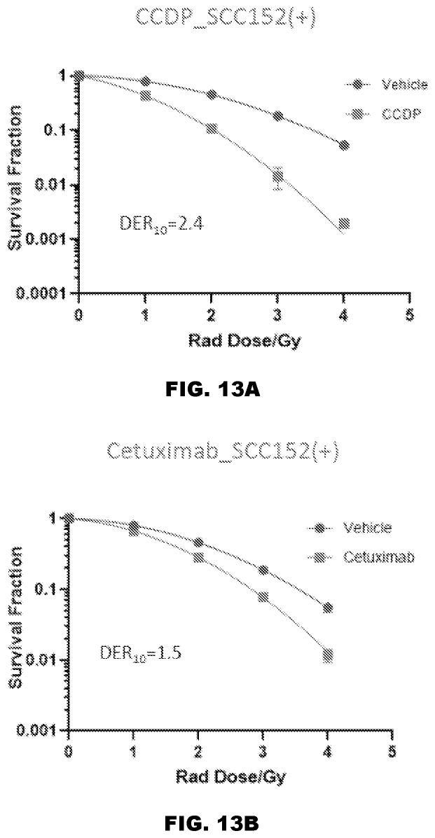

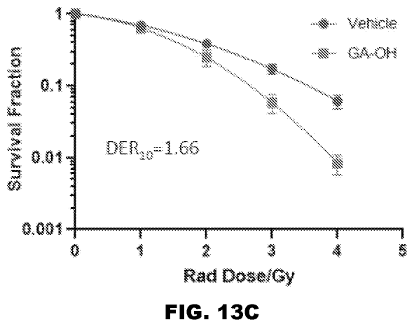

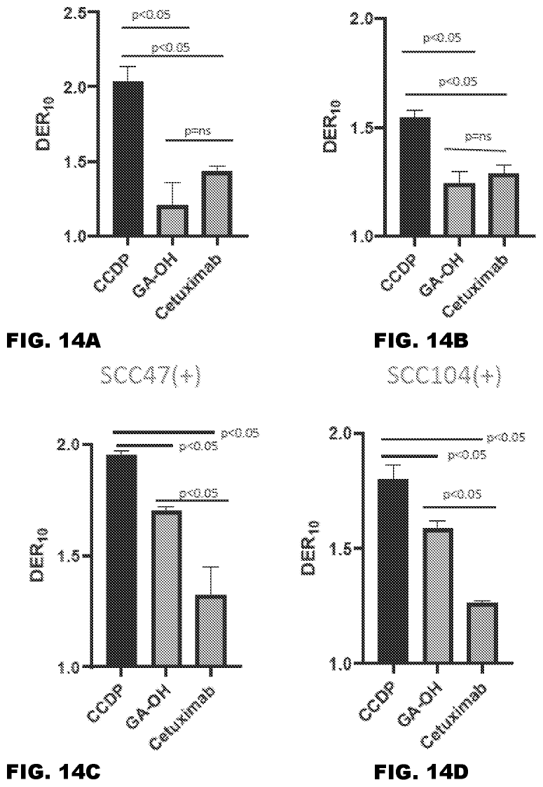

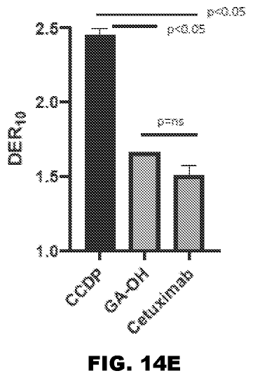

Sensitization of an additional HPV⁺ cell line to radiation by CDDP, Cetuximab and GA-OH was evaluated. FIGS. 13A-13C are graphical representations of the radio-response of HPV⁺ cell line (SCC152) when subject to photon radiation followed by treatment with either the vehicle alone or cisplatin or cetuximab or GA-OH, as measured by MTT assays. GA-OH enhanced the sensitization of this HPV⁺ cell line also to photon radiation. FIGS. 14A-14E are graphical representations of the extent of sensitization in HNSCC cell lines as measured by DER₁₀ when HPV⁻ cell lines (SCC19 and SCC29) and HPV⁺ cell lines (SCC47, SCC104, and SCC152) are subject to photon radiation followed by treatment with cisplatin or cetuximab or GA-OH. Generally, CDDP shows the greatest sensitization in both HPV⁺ and HPV⁻ cell lines and cetuximab shows the least sensitization. GA-OH shows sensitization in HPV⁺ cell lines. Its magnitude of sensitization is less than that of CDDP but greater than cetuximab.

### GA-OH Affects the Cell-Cycle Response in HPV⁺ Cell Line to Radiation

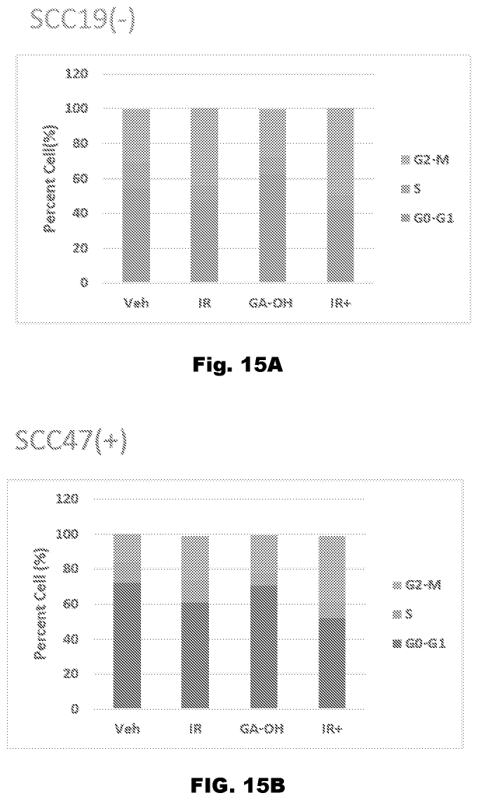

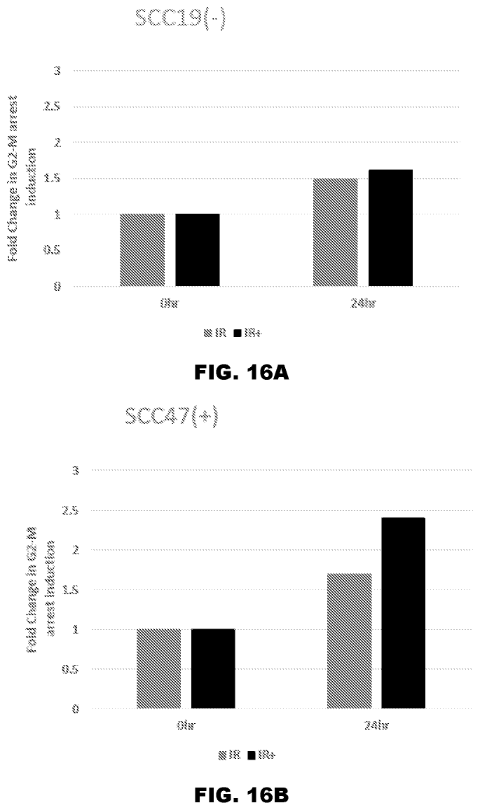

Cells were treated with radiation (4Gy) followed by GA-OH before cell cycle analysis was performed using propidium iodide (PI) flow cytometry assay after 24 hours. FIGS. 15A-15B are graphical representations of the cell cycle analysis of SCC47 and SCC19 cell lines after irradiation and followed by treatment with control or with GA-OH, as measured by flow cytometry assays after 24 hours. In both cell lines, G2-M is the most affected stage of cell cycle and there is increase in G2-M arrested cells. The HPV⁺ cell line—SCC47 has a higher proportion of cells that are still arrested in G2-M phase after 24 hours compared to the HPV⁻ cell line—SCC19. FIGS. 16A-16B are graphical representations of the fraction of G2-M arrested cells in SCC47 and SCC19 lines after irradiation and followed by treatment with control or with GA-OH, as measured by flow cytometry assays after 24 hours. Higher percentage of cells arrested in SCC47 compared to SCC19. In the HPV⁺ but not HPV⁻ line, the addition of GA-OH to the radiation protocol increased arrest at 24 hours as compared to radiation alone.

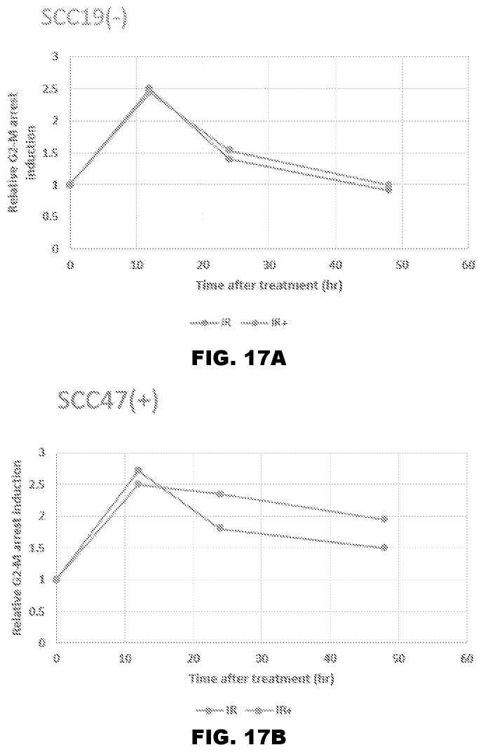

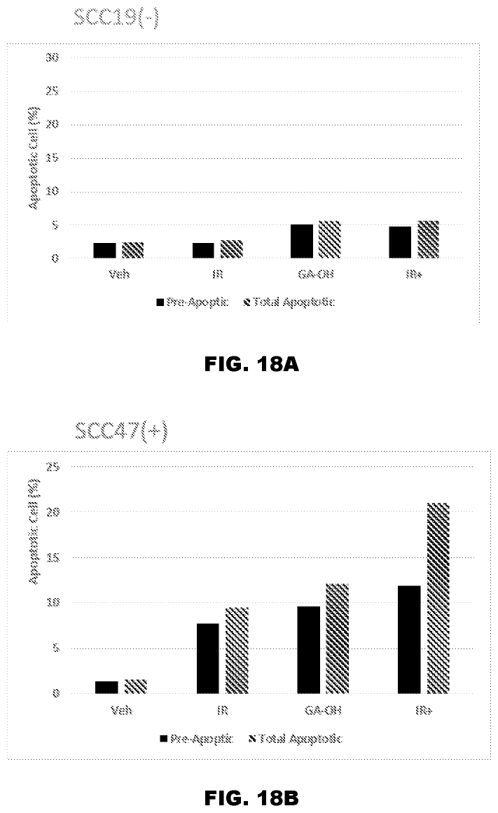

Kinetics of G2-M arrest when SCC47 and SCC19 are irradiated with or without GA-OH. Cells were treated with radiation (4Gy) followed by GA-OH before cell cycle analysis was performed using Annexin V flow cytometry assay after 24 hours. FIGS. 17A-17B are graphical representations of the kinetics of G2-M arrest when SCC47 and SCC19 are irradiated and followed by treatment with control or with GA-OH, as measured by flow cytometry assays after 24 hours. Cells are arrested to the same extent in the short term (first 12 hours) but over extended periods, SCC19 cells exit the G2-M stage quicker. In the HPV⁺ but not HPV⁻ line, the addition of GA-OH to the radiation protocol delays the exit from the exit the G2-M stage at 24 hours as compared to radiation alone. Apoptosis analysis of SCC47 and SCC19 was performed after irradiation and followed by treatment with control or with GA-OH, as measured by flow cytometry assays after 24 hours. FIGS. 18A-18B are graphical representations of the apoptotic analysis of SCC47 and SCC19 after irradiation with or without GA-OH after 24 hours, as measured by flow cytometry assays. Radiation induces minimal apoptosis on its own. GA-OH induces appreciable apoptosis alone. Combination of radiation with GAOH induces greater apoptosis and at a greater rate in HPV⁺ cell line—SCC47 compared to HPV⁻ cell line—SCC19.

### Materials and Methods

### Purification and Preparation of Proteins

Plasmids carrying E6 and caspase 8 (pGEX-E6 and pTriEx-Caspase 8) were previously constructed. Expression of GST-E6, GST-Caspase 8 and His₆-Caspase 8 in E. coli and subsequent purification were carried out as previously described. GST-E6, GST-Caspase 8 and His₆-Caspase 8 proteins were diluted into GST protein buffer (PBS pH 8.0, 5% glycerol, 2 mM DTT) and His protein buffer (20 mM HEPES pH 7.4, 150 mM NaCl, 2 mM KCl, 5% glycerol, 2 mM DTT), respectively. The concentration of the proteins was determined using Coomassie Plus—The Better Bradford Assay Reagent (Thermo Scientific, Waltham, MA, USA). Purity of the isolated proteins was assessed by sodium dodecyl sulfate-polyacrylamide gel electrophoresis (SDS-PAGE) separation and Coomassie staining.

### Compound Library Collection

The library used for the screening comprised 3 sub-libraries for a total of about 5040 small molecule compounds. The Prestwick Chemical Library contains 1200 small molecules. Many compounds in this library possess drug-likeness properties (bioavailability and safety in humans), because 90% of the compounds are previously or currently marketed drugs, while 10% are bioactive alkaloids or related substances. The Microsource Spectrum Collection consisted of 2000 small molecules with a wide range of biological activities and structural diversity. Some of the compounds were known drugs, while others were natural products and non-drug enzyme inhibitors with pharmacological profiles not yet well characterized. The remainder were synthetic compounds that were uniquely synthesized by the Kansas University Chemistry Core as well as the Center for Chemical Methodology and Library Methodology.

### Primary Library Screening and Initial Hit Selection

In total, the collection of compounds contained 5040 small molecules from 3 structurally diverse compound libraries (see Table 3 for more details). The compounds were diluted to a working concentration in DMSO and screened at a single-point final concentration of 10 μM with no replicates. Briefly, 75 nL of each compound was transferred and added to wells of the destination 384-well plate using the Echo dispenser to 4 μL of blocking solution. 4 μL of 800 nM GST-E6 and 4 μL of His₆-Caspase 8 were added and pre-incubated at room temperature for 60 minutes. 8 μL of the donor and acceptor bead mixture (final concentration of 20 μg/ml) was then added. The plates were sealed and incubated for 4 hours at room temperature before the plates were read using the Envision™ Multi-Label plate reader (Perkin Elmer Inc.). Percent inhibition for each compound was calculated, and the % inhibition value that was 3 standard deviations (SD) above the sample mean (μ+3SD) was used as the selection threshold. A 10-point serial dilution of these compounds was done for dose-dependency reconfirmation. Dose response inhibition curves were constructed and IC₅₀ calculated using GraphPad Prism using four parameter non-linear regression analysis.

TABLE 3
Content of the 3 libraries used in the screen.

| Library | No. of compounds | Tanim [symbol] ndex | Type of compounds | Description |
|---|---|---|---|---|
| CMLD | 1840 | 0. [symbol] 02 | Diversity, drug-like | Diverse 3D pharmacophores |
| | | | | used for selection. Over [symbol] |
| | | | | proprietary chemical filters |
| | | | | applied (including Lipinski's |
| | | | | rule of 5) and Daylight |
| | | | | Tanimoto similarity measures |
| | | | | to ensure structural diversity |
| | | | | and drug-likeness. |
| Pres [symbol] wick | 1200 | 0.81 | FDA Approved drugs | Marketed drugs |
| Microsource spectrum | 2000 | 0.80 | Bioactives (5 [symbol] 0 | 75% of the natural product |
| | | | compounds), FDA | collection encompasses: |
| | | | approved (640 drugs), | alkaloids (16%), flavanoids |
| | | | Natural products (800 | (12%), sterols/triterpenes (12%) |
| | | | compounds) | diter [symbol] enes/sesqu [symbol] erpenes: 10%) [symbol] |
| | | | | benzophenones/chalcones/ |
| | | | | [symbol] benes (10%) [symbol] |
| | | | | limonoids/quassinoids (9%) [symbol] |
| | | | | and chromones/coumar [symbol] |
| | | | | (6%). The remainder 25% of |
| | | | | the collection includes includes [symbol] |
| | | | | quinones/quinonemethides [symbol] |
| | | | | benzofurans/benzopyrans, |
| | | | | [symbol] otenoids/xanthones, |
| | | | | carbohydrates, and |
| | | | | benz [symbol] ropolones/depsides/ |
| | | | | depsidones, in descending order. |

[symbol]

indicates data missing or illegible when filed

### Counter-Screen Assays and Hit Confirmation

The first counter-screen assay was based using the GST-His₆ fusion peptide as the E6-binding partner instead of GST-E6-His₆-Caspase 8. Hit candidate compounds from the primary screen were prepared using a 6-point serial dilution. Using an Echo dispenser, compounds were transferred to plates containing 4 μL of blocking buffer. 8 μL of 5 nM GST-His₆ peptide substrate was then added. The mixture was pre-incubated at room temperature for 60 minutes. Glutathione donor and nickel chelate acceptor beads (final concentration 20 μg/mL) were added and incubated for another 60 minutes at room temperature. Dose-dependency of the compounds using GST-E6-His₆-Caspase 8 was performed in parallel using the same protocol as in the primary screen. The signals were then read using the Envision™ plate reader. Following IC₅₀ calculations using GraphPad Prism, selection was based on the Selectivity Index (SI) and a maximum inhibition of E6-caspase 8 binding ≥50%.

The second counter-screen was based on GST-Caspase 8 and His6-Caspase 8. Hits from the GST-6xHis counter-screen were tested in triplicate at a single concentration of 10 μM. Briefly, 5 μL of the compound was manually added to the plate wells containing 5 μL blocking buffer. 5 μL of 400 nM GST-Caspase 8 and 5 μL 400 nM His₆-Caspase 8 were added and pre-incubated for 1 hr at room temperature. Glutathione donor and nickel chelate acceptor beads (final concentration 20 μg/mL) were added and incubated for another 60 minutes at room temperature before signal was quantified. This experiment was repeated 2 times on different days. Results were processed as described above, and % inhibition was calculated relative to the vehicle control. Compounds with % inhibition of caspase 8 dimerization less than 20% were chosen for further consideration.

### Cheminformatic Filtering and Cross-Validation

Compounds that passed the two counter-screens were subjected to cheminformatic analysis as an additional filter for recognition and exclusion of compounds with problematic substructures. These substructures contain functional groups that may disrupt binding in many unrelated biochemical assays in a non-specific manner. Specifically, from the names and SMILES of the hit compounds, the following databases were queried to find hits with pan assay interference (PAINS) patterns: Zinc15, SwissADME, FAFdrugs4 and PAINS-Remover. Compounds that made it onto the consensus list as having no PAINS patterns after filtering with these online tools were then selected. The selected compound(s) were then cross-validated with a related but different primary screen assay. Specifically, the caspase 8 used in the primary screen was replaced with E6AP and the inhibitory activity was evaluated against E6 binding to E6AP (E6-E6AP) using the same steps as in the AlphaScreen™ protocol described above.

### Structure Activity Relationships (SARs)

Using the SciFinder and Zinc15 databases, several gambogic acid structural analogs were identified and selected. The 8 analogs were obtained as follow: gambogenic amide (Enzo Life Sciences), gambogenic acid (Selleckchem), morellic acid (Aobious), 30-hydroxy gambogic acid (Quality Phytochemicals, LLC), acetyl gambogic acid (Microsource), and gambogin, neogambogic acid and isomorellinol (MolPort Natural Products). Additional gambogic acid was purchased from Tocris. The interactions of the analogues with E6-caspase 8 was tested using AlphaScreen™ technology using the same protocol as with the primary screen, and were compared to the parent compound. The effect on cell viability was also similarly done in HPV⁺ and HPV⁻ cell lines via the MTT assay (see below) and potency was determined using GraphPad IC₅₀ curve fitting.

### Cell Culture

Saos-2, SiHa, and CaSki cells were obtained from the America Type Culture Collection (Manassas, VA, USA). SiHa and CaSki were cultured in Eagle's minimal essential medium (Invitrogen, Carlsbad, CA, USA) as described previously. HNSCC cell lines were obtained from several sources: UM-SCC47-TC-Clone 3 (#47CL3), UPCI-SCC90-UP-Clone 35 (#90), and SCC 84 were a gift from Dr. John Lee, Sanford Research (South Dakota, USA). UMSCC 19 (#19), UMSCC 29 (#29), UMSCC49 (#49) and UMSCC 104 (#104) were a gift from Dr. Thomas Carey, University of Michigan (Michigan, USA). UPC1-SCC152 was purchased from ATCC. HNSCC cells were cultured in Dulbecco's Modified Eagle Medium (Mediatech, Manassas, VA, USA) supplemented with 10% of FBS. Saos-2 cells were grown in McCoy 5a medium, and HCT116 cells were cultured in RPMI medium supplemented with 10% FBS.

### MTT Cell Viability Assays

All working concentrations were diluted in PBS to the desired concentration before use. To test the effect of gambogic acid and/or its derivatives on cell viability, all cell lines were seeded at 2×10⁴ per well in 96-well plates and allowed to adhere overnight. Various concentrations of the analogues were added and the cells incubated at 37° C. for 24 hr. Viability was then measured using the MTT assay, performed as described previously. All experiments were repeated at least three times (three biological replicates, carried out on different days). Data presented are from a representative experiment. Cell viability and potency were assessed from % inhibition relative to the vehicle control, and IC₅₀ dose curves were generated using GraphPad Prism.

### Caspase Activity Assay

Cells were seeded into white walled 96-well plates at 2×10⁴ cells per well in 100 μL media and incubated overnight. GA-OH (0.75 μM) and vehicle were then added and incubated at 37° C. for 24 hr. Caspase 3/7 activity was measured using the Caspase 3/7 Glo kit (Promega, Fitchburg, WI, USA) following the manufacturer's instructions. Briefly, room temperature-equilibrated Caspase-Glo reagent was added (Promega) to each well. The plate was mixed by placing it on an orbital shaker and incubated for 30 secs at room temperature, then incubated at room temperature. After a 2-hr incubation, luminescence was measured using a plate-reading fluorimeter (Flx800, Bio-Tek Instrument Co., Winooski, VT, USA). Background activity (blank reaction) was subtracted from all experimental wells. Percent activity of caspase 3/7 in wells treated with GA was then expressed relative to vehicle treated wells.

### Western Blotting

Adherent cells were washed with ice cold PBS. Cell lysis buffer containing protease inhibitor cocktail was added and cells were scraped off into a tube on ice. The cells were incubated on ice for 10 minutes. Cell lysates were separated by SDS-PAGE and electrophoretically transferred to PVDF membranes. Following blocking, antibodies directed against caspase 8, p53, cleaved PARP, cleaved caspase 3, p21, and β-actin (Cell signaling) were applied at 1:5000 dilution. Anti-mouse and anti-rabbit secondary antibodies were then employed (LI-COR Biosciences, Lincoln, NE, USA). Signals were measured using the Odyssey Infrared Imaging system (LI-COR Biosciences) and quantified using Image J.

### Colony Formation Assay

Sub-confluent monolayer cells were treated with different doses of GA-OH for 24 hours. Cells were trypsinized and re-suspended before re-plating into 6 well plates in DMEM or MEM at 500-1000 cell densities, depending on the cell line. Cells were then allowed to grow for 10-20 days, depending on the cell line, before fixing and staining. A mixture of methanol/acetic acid was used for fixing, followed by 0.5% crystal violet staining. Plates were imaged using UV imager, and colonies with more than 50 colonies counted using image J. Surviving fractions were determined by dividing the number of colonies by the number of cells seeded as a product of the corresponding plating efficiency. Survival fractions curves were plotted using GraphPad Prism.

### Data Analysis

Binding and dose-response curves were fitted using GraphPad software (GraphPad Software, Inc., La Jolla, CA).

Z-factor was calculated from intraplate controls as previously described using the formula [29]: Z′=1−(3*STDEV_{Control}+3*STDEV_{Background})/(Mean_{Control}−Mean_{Background}) where STDEV is the standard deviation and control is 0% inhibition (maximum signal) and background is 100% inhibition (minimum signal).

Signal to background ratio was determined as follows: S/B ratio=Mean_{control}/Mean_{background}

Percent (%) activity and Percent (%) inhibition of binding for the compounds was calculated from Alpha Screen signals using the equations:

$$Percent ( \% ) activity : 100 ( {Mean}_{compound} - {Mean}_{background} ) / ( {Mean}_{control} - {Mean}_{background} ) \\ Percent ( \% ) inhibition : 100 - \% activity . \\ Percent Coefficient of Variation : ( CV \% ) = 100 * ( STDEV / Mean ) \\ Selectivity Index ( SI ) : IC {50}_{GST - His peptide} / IC {50}_{E 6 - Caspase 8} >= 10 \\ Hit Selection Threshold : \geq μ + 3 SD where μ is the sample mean and SD is standard deviation$$

In certain embodiments, compounds described here can be developed for further potency or targeted delivery. Further modifications and alternative embodiments of various aspects of the compositions and methods disclosed here will be apparent to those skilled in the art in view of this description. Accordingly, this description is to be construed as illustrative only and is for the purpose of teaching those skilled in the art the general manner of carrying out the embodiments. Elements and materials may be substituted for those illustrated and described here, parts and processes may be reversed or omitted, and certain features of the embodiments may be utilized independently, all as would be apparent to one skilled in the art after having the benefit of this description of the embodiments. Changes may be made in the elements described here without departing from the spirit and scope of the embodiments as described in the following claims.

What is claimed is:

1. A method of treating a human papillomavirus-associated cancer in a subject, the method comprising administering to the subject a therapeutically effective amount of gambogic acid or a pharmaceutically acceptable derivative thereof.

2. The method of claim 1, wherein the human papillomavirus-associated cancer is one of a cervical cancer, an oropharyngeal cancer, an anal cancer, a penile cancer, a vaginal cancer, or a vulvar cancer.

3. The method of claim 1, wherein the human papillomavirus-associated cancer is head and neck squamous cell carcinoma.

4. The method of Claim 1, further comprising administering to the subject a therapeutically effective amount of an apoptosis-activating chemotherapeutic agent.

5. The method of Claim 1, further comprising administering to the subject a therapeutically effective amount of one or more of cisplatin, cetuximab, and doxorubicin.

6. The method of Claim 1, further comprising administering to the subject a therapeutically effective amount of radiation prior to the administration of the therapeutically effective amount of gambogic acid or a pharmaceutically acceptable derivative thereof.

7. The method of Claim 1, further comprising administering to the subject a therapeutically effective amount of radiation subsequent to the administration of the therapeutically effective amount of gambogic acid or a pharmaceutically acceptable derivative thereof.

8. The method of Claim 6, further comprising administering to the subject a therapeutically effective amount of an apoptosis-activating chemotherapeutic agent.

9. The method of Claim 6, further comprising administering to the subject a therapeutically effective amount of one or more of cisplatin, cetuximab, and doxorubicin.

10. A method of treating a human papillomavirus-associated cancer in a subject, the method comprising administering to the subject a therapeutically effective amount of 30-hydroxygambogic acid or a pharmaceutically acceptable derivative thereof.

11. The method of claim 10, wherein the human papillomavirus-associated cancer is one of a cervical cancer, an oropharyngeal cancer, an anal cancer, a penile cancer, a vaginal cancer, or a vulvar cancer.

12. The method of claim 10, wherein the human papillomavirus-associated cancer is head and neck squamous cell carcinoma.

13. The method of Claim 10, further comprising administering to the subject a therapeutically effective amount of an apoptosis-activating chemotherapeutic agent.

14. The method of Claim 10, further comprising administering to the subject a therapeutically effective amount of one or more of cisplatin, cetuximab, and doxorubicin.

15. The method of Claim 10, further comprising administering to the subject a therapeutically effective amount of radiation prior to the administration of the therapeutically effective amount of 30-hydroxygambogic acid or a pharmaceutically acceptable derivative thereof.

16. The method of Claim 10, further comprising administering to the subject a therapeutically effective amount of radiation subsequent to the administration of the therapeutically effective amount of 30-hydroxygambogic acid or a pharmaceutically acceptable derivative thereof.

17. The method of Claim 15, further comprising administering to the subject a therapeutically effective amount of an apoptosis-activating chemotherapeutic agent.

18. The method of Claim 15, further comprising administering to the subject a therapeutically effective amount of one or more of cisplatin, cetuximab, and doxorubicin.
